(19) United States
(12) Patent Application Publication
(10) Pub. No.: US 2026/0234629 A1
(43) Pub. Date: Aug. 13, 2026

(54) ANTISENSE MOLECULES AND THEIR USES FOR THE TREATMENT OF CORONAVIRUS INFECTION, IN PARTICULAR THE TREATMENT OF COVID-19 RELATED TO SARS-COV-2 INFECTION

(71) Applicants: Institut National De Recherche Pour L'Agriculture, L'Alimentation Et L’Environnement, PARIS (FR); INSTITUT NATIONAL DES SCIENCES ET INDUSTRIES DU VIVANT ET DE L'ENVIRONNEMENT, PALAISEAU (FR); UNIVERSITE DE VERSAILLES SAINT-QUENTIN-EN-YVELINES, VERSAILLES (FR); ÉCOLE NATIONALE VETERINAIRE D'ALFORT, MAISON-ALFORT (FR); UNIVERSIDAD ANDRES BELLO, SANTIAGO (CL)

(72) Inventors: ERIC BARREY, BARBIZON (FR); CHRISTOPHER SHAWN FITZPATRICK MARIN, SANTIAGO (CL); SOPHIE POLLET, SAINT REMY LES CHEVREUSE (FR); BERNARD DELMAS, BOURG LA REINE (FR); NICOLAS MEUNIER, ORSAY (FR); BRUNO DA COSTA, ST CHERON (FR); JEAN FRANÇOIS ÉLÉOUËT, BREUILLET (FR); SOPHIE LE PODER, SAINT-MAUR (FR); VERÓNICA ANDREA BURZIO MENENDEZ, SANTIAGO (CL)

(21) Appl. No.: 19/147,811

(22) PCT Filed: Jan. 15, 2024
(86) PCT No.: PCT/EP2024/050800

§ 371 Date: Jul. 14, 2025

(30) Foreign Application Priority Data
Jan. 16, 2023 (EP) 23305055.8

(51) Int. Cl. C12N 15/113; A61K 31/7115; A61K 31/712; A61K 31/7125; A61P 31/14

(52) U.S. Cl. CPC C12N 15/1131; A61K 31/7115; A61K 31/712; A61K 31/7125; A61P 31/14; C12N 2310/11; C12N 2310/3145; C12N 2310/315; C12N 2310/3181; C12N 2310/321; C12N 2310/3231; C12N 2310/3341; C12N 2310/335; C12N 2310/346

(57) ABSTRACT

The present invention relates to a new antisense oligonucleotide, a pharmaceutical composition comprising it and their use for preventing or treating infection by coronavirus, especially by SARS-CoV-2 virus which causes the infection disease COVID-19.

### FIELD OF THE INVENTION

The present invention relates to field of the medicine, especially the prophylaxis and treatment of coronavirus infections. It relates to antisense oligonucleotide molecules directed against coronavirus, and their uses for the treatment of coronavirus infection, especially infection by SARS-CoV-2 which causes the infection disease COVID-19.

### BACKGROUND OF THE INVENTION

Recently, the world has been dealing with outbreaks of a human-infecting betacoronavirus, which spread globally and caused a global pandemic, responsible for the severe acute respiratory syndrome coronavirus (SARS-CoV-2).

The disease is responsible for causing various serious symptoms in the population, including fever, cough, shortness of breath, fatigue, headache, loss of smell (anosmia), nasal congestion, sore throat, coughing up sputum, pain in muscles or joints, chills, nausea, vomiting, and diarrhea. In severe cases, symptoms can include difficulty waking, confusion, blueish face or lips, coughing up blood, decreased white blood cell count, kidney failure, biochemical parameters and other signals related to the physiopathology. Complications can include pneumonia, viral sepsis, acute respiratory distress syndrome, and kidney failure with potential lethal outcome. As of now, WHO has reported over 655 million cases with no less than 6.67 million deaths worldwide (Dec. 22, 2022).

Affections linked to SARS-CoV-2 have been a growing issue to public health services in recent years, with the virus being highly contagious and unpredictable, between asymptomatic and symptomatic carriers, leading some individuals to require proper hospitalization and the dire assistance of healthcare professional, outrun by pandemic-level numbers of patients.

Successful development of vaccines, especially the newly emerged mRNA-based vaccines, led to establish a control over the pandemic. However, antiviral drugs are strongly needed to complement vaccines in clinics when patients are already infected, or as prophylactics for high-risk groups and to control any future outbreaks of coronaviruses.

In that regard, the potential of small molecule drugs that target viral RNA components and/or the genomic RNA as antiviral agents has been widely recognized.

To that extent, use of Antisense Oligonucleotides (ASOs), which are short, single-stranded nucleic acids molecules (most of the time DNA or RNA after modifications), has proven useful to interact and cleave with messenger RNA or non-coding RNA (ncRNA) to prevent translation of a targeted gene or ncRNA regulatory activity. Their DNA sequence is complementary to the specific RNA target and their binding leads to degradation of the RNA sequences with failure of protein production. Accordingly, ASO therapy has so far been proposed for the treatment of rare diseases, metabolic diseases, neurodegenerative diseases, cancer, and pathogenic infections.

SARS-CoV-2 is positive-sense single-stranded RNA virus with a viral genome (genomic RNA) of approximately 30 kilobases (kb) containing sequences that can make excellent target viral regions, such a 5′ untranslated region (5′UTR), ORF1ab region, encoding non-structural viral proteins, the downstream 3′ segment, with sequences for the structural proteins (including spike(S), envelope (E), membrane (M) and nucleocapsid (N)) and a 3′ untranslated region (3′UTR).

In this context, there were several attempts to develop ASO molecules targeting SARS-CoV-2. Two reviews provide a summary of these ASO molecules and their effects (Hedge et al, 2021, Frontiers in Chemistry, doi: 10.3389/fchem.2021.802766; Quemener and Galibert, 2021, WIREs RNA. 2022; 13: e1703, DOI: 10.1002/wrna.1703). Hedge et al discloses ASOs targeting 5′ UTR region, ORF1a and s2m sequences of the 3′ UTR region. Quemener and Galibert discloses ASOs targeting 5′ UTR region and ORF1a. Zhu et al. (2022, Nature Communications, 13, 4503; doi: 10.1038/s41467-022-32216-0) discloses intranasal LNA modified therapeutic ASO targeting SARS-CoV-2. ASO targeting 5′ leader sequence, 5′ UTR region, FSE region of ORF1ab and S gene have been tested. ASOs targeting the 5′ leader sequence have been identified as the most effective, as they interfere with the formation of SL1 stem-loop structure in the 5′ leader sequence and disrupt the secondary structure of the SL1, which protects the viral RNA from Nsp1-mediated translational suppression.

Vora et al. (2022, PNAS; 119, e2117198119; doi: 10.1073/pnas.2117198119) discloses ASOs targeting 5′ UTR region of SARS-CoV-2, and more specifically SL1 loop of 5′ UTR region. Targeting SL1 here aims at a similar purpose disclosed in Zhu et al, to inhibit the viral translation and make SARS-CoV-2 vulnerable to Nsp1 suppression, with a high degree of conservation among variants.

Li et al (2021, bioRxiv doi: 10.1101/2021.08.23.457434) discloses ASOs targeting FSE region in ORF1 and TRS region in the 5′-UTR of SARS-CoV-2. Zhang et al (2021, Nat Struct Mol Biol, 9:747-754. doi: 10.1038/s41594-021-00653-y) discloses ASOs targeting FSE region of SARS-CoV-2.

Lulla et al (2021, Journal of Virology, 95, e00663-21; doi: 10.1128/JVi.00663-21) discloses ASOs targeting the s2m element of the 3′ UTR of SARS-CoV-2. s2m targets are highly conserved, highly important/functional but not highly repeated sequences.

Dauksaite et al (2022, bioRxiv, doi: 10.1101/2022.11.28.518195) discloses multiple ASO molecules targeting SARS-CoV-2, in particular ORF1a, ORF1b, N and 5′ UTR, and identified an ASO molecule with the highest efficiency targeting the 5′ UTR and the N regions, for the prevention and treatment of SARS-CoV-2 infections.

Dhorne-Pollet et al (Front Microbiol. 2022, 13:915202. doi: 10.3389/fmicb.2022.915202) discloses multiple ASO molecules targeting SARS-CoV-2, in particular ORF1a, ORF1b, N and the 5′ UTR region, and an ASO molecule with the highest efficiency targeting ORF1b. ASO molecules targeting the 5′ UTR and the N regions are less efficient for inhibiting the viral replication.

In addition, WO2022/031410 discloses ASOs targeting SARS-CoV-2, especially 5′ leader sequences, 5′ UTR, FSE of ORF1a/b and Spike coding region. WO2021/207641 and WO2021/195025 also disclose ASOs targeting SARS-CoV-2.

However, the need of efficient ASOs or combinations of ASOs against SARS-CoV-2 infection remains for treatments of patients infected by emerging SARS-CoV-2 variants before hospitalization and intensive care.

### SUMMARY OF THE INVENTION

The present invention provides a new antisense oligonucleotide and pharmaceutical compositions comprising it, and their use to treat or prevent coronavirus infection, such as infection by SARS-CoV-2. More particularly, the inventors identified that one ASO targeting a specific genome sequence of SARS-CoV-2 decreases the viral RNA load and associated viral particles in vitro and in vivo to very high levels. Without wishing to be bound by theory, the inventors claims that the high efficiency of this ASO could be partially due to i) the good choice of the main target sequence of the genomic RNA (key function, ASO accessible sites, binding affinity); ii) the multiple secondary repeated target sequences of this ASO present in the SARS-CoV-2 genome for blocking/cleaving several regions; iii) the combination of two (or more) ASOs targeting close or different regions of the genomic RNA; and iv) these combination of ASOs can consist of ASOs made with different chemical modifications for simultaneously blocking and cleaving the RNA target sequence. Additionally, the target site of the ASO of the present invention actually shows a great percentage of identity among all recent SARS-CoV-2 variants of concern (VOC), promising great results on past and future SARS-CoV-2 infections. Potential routes for administration include intranasal way, which has proven to be much easier and quicker for patients, with potential for low dosage forms positively balanced with more efficiency and less toxicity.

The inventors demonstrated that an ASO (oligonucleotides RNase H1 dependent) is much more efficient at downregulating the targeted RNA than an siRNA designed to target the same sequence. The inventors hypothesize is that the higher efficiency is due to a lower saturation of the mechanism of action. In fact, siRNA silencing involves microRNA maturation pathway which involves a significant number of steps and depends on the presence and activity of multiple protein complexes. An ASO involves fewer steps and the risk of saturation is therefore lower. In addition, the inventors decided to combine ASOs with different chemistries that have complementary effects (cleaving and blocking) to further reduce the risk of saturation. They confirmed their hypothesis and observed a synergistic effect when ASOs that act via RNase H1 mediated degradation by cleaving and ASOs (MPO and PNA) that act by blocking the target sequence are combined.

Therefore, the present invention relates to an ASO, wherein the ASO comprises, essentially consists in or consists in a sequence set forth in SEQ ID NO: 1. Preferably, the ASO comprises or consists in a sequence of SEQ ID NO:1.

Optionally, the ASO of the invention comprises one or several chemical modifications, preferably modifications of nucleotide and/or modifications of internucleotide linkage, in particular in order to increase stability, affinity for the target sequence and delivery to the infected cells. Optionally, the ASO acts via RNase H1 mediated degradation. Optionally, the ASO acts via blocking the target sequence. In a preferred aspect, the ASO of the invention comprises internucleotide linkage modification with phosphorothioate (PS) linkage modification, preferably at least for 3 to 5 terminal 5′ and/or 3′ nucleotides. Optionally, all the internucleotide linkages of the ASO are phosphorothioate (PS) linkages.

In addition or alternatively, the ASO comprises nucleotide modifications selected from locked nucleic acid (LNA) modification, 2′-O-methoxyethyl(2′-O-MOE) modification, 2′-O-methyl(2′-O-Me) modification, 2′-fluoro (2′-F) modification, phosphorodiamidate morpholino oligomer (PMO) modification, peptide-conjugated phosphoroamidate morpholino oligomer (PPMO) modification, peptide nucleic acid modification (PNA), constrained ethyl (cEt) bridged nucleic acid (BNA) modification, tricyclo-DNA (tcDNA) modification and 5-methyl (5-M) modification or combinations thereof. Preferably, the ASO of the invention comprises nucleotide modifications selected from the group consisting of LNA modifications, preferably at least for 3′ and 5′ ends of ASO sequence, more preferably at least for 3 to 5 terminal 5′ and/or 3′ nucleotides.

The 2′-O-MOE modifications and 2′-O-Me modifications are optionally placed on one or more nucleotides of the ASO sequence.

Optionally, the ASO of the invention comprises 5-methyl cytosine, and/or 5-methyl uracil modifications. In a very particular aspect, the ASO of the invention comprises nucleotides which all are phosphorodiamidate morpholino oligomer (PMO). In another very particular aspect, the ASO of the invention comprises nucleotides which all are peptide nucleic acid oligomer (PNA).

Optionally, the ASO is selected from an ASO as described in any of SEQ ID NOs: 1, 24, 45-103 and 105, preferably 1, 24, 45-101 and 105.

The invention also relates to a pharmaceutical composition which comprises the ASO according to the present invention and optionally a pharmaceutically acceptable carrier.

Optionally, the pharmaceutical composition of the invention further comprises one or more ASO complementary to SARS-CoV-2 genome, especially ASO having a target site in 5′ UTR, ORF1a, ORF1b, S, ORF3a, E, M, ORF6, ORF7a, ORF7b, ORF8, N, ORF10, or 3′ UTR regions, more preferably in 5′UTR, ORF1ab-nsp1, ORF1ab-nsp12, ORF1ab-FSE, or N. Preferably, the pharmaceutical composition of the invention further comprises one or more ASO selected from the group consisting of the ASO comprising, essentially consisting in or consisting in a sequence set forth in SEQ ID NOs: 2 to 21, preferably SEQ ID NOs: 2 to 6, more preferably SEQ ID NOs: 2 to 4. More preferably, the pharmaceutical composition of the invention further comprises one ASO comprising, essentially consisting in or consisting in a sequence set forth in SEQ ID NO: 2, SEQ ID NO: 3, SEQ ID NO: 4, or SEQ ID NO: 19.

In a preferred aspect, the invention relates to a pharmaceutical composition comprising two or more ASOs with different chemistries that exhibit complementary effects (cleaving and blocking). Optionally, the pharmaceutical composition comprises one ASO comprising phosphorothioate (PS) linkage modification and/or nucleotide modifications selected from the group consisting of Locked Nucleotide Acid (LNA) modifications and 2′-O-MOE modifications and one ASO in which all nucleotides are phosphorodiamidate morpholino oligomer (PMO) or peptide nucleic acid (PNA). Optionally, the ASOs of the pharmaceutical composition comprise, essentially consist in or consist in a sequence set forth in SEQ ID NO: 1. Optionally, the pharmaceutical composition comprises one ASO selected from the group consisting of SEQ ID NOs 24, 45-101 and 105, and one ASO of selected from the group consisting of SEQ ID NOs 102 and 103.

In addition, the invention relates to an ASO or a pharmaceutical composition according to the disclosure for use as a drug. More specifically, the invention relates to an ASO or a pharmaceutical composition according to the disclosure, for use in the treatment or prevention of a viral infection by a coronavirus, preferably an infection by a severe acute respiratory syndrome-related coronavirus (SARS-CoV), more preferably a SARS-CoV-2 or MERS-CoV virus infection.

Optionally, the ASO or pharmaceutical composition is formulated for injection such as SC (sub-cutaneous), IP (intraperitoneal) or IV (intravenous), or preferably for inhalation or nebulization, preferably an intranasal or mouth inhalation or nebulization.

### BRIEF DESCRIPTION OF THE FIGURES

FIG. 1: Comparison of ASOs designed to target 5′UTR, ORF1ab, N, or 3′ UTR of the SARS-CoV-2 genome for their effects on SARS-CoV-2 inhibition measured by N-CoV RT-qPCR.

The efficiency to inhibit the SARS-CoV-2 was evaluated by RT-qPCR to measure N-CoV RNA quantity in HEK-293T/ACE2 cells in culture. The quantity of viral RNA copies (N-CoV) reached 94% reduction with the ASO-29391 compared to ASO-C.

FIG. 2: Comparison of ASOs designed to target 5′UTR region, nucleocapsid gene and 3′UTR region for their effects on SARS-CoV-2 inhibition by minigenome reporter.

The efficiency to inhibit the SARS-CoV-2 was evaluated by measuring the luminescence by co-transfection of ASOs and the minigenome luciferase reporter including only the 5′UTR, nucleocapsid coding gene and 3′UTR regions of SARS-CoV-2 genome in transfected cells in culture. The quantity of the firefly luciferase activity reached 95% reduction with the ASO-29391 compared to ASO-C.

FIG. 3: Combined pairs of ASOs better inhibit SARS-CoV-2 replication than the same single ASOs

This figure shows the relative expression to the control ASO-C of the viral N-CoV transcript in cells infected and transfected either by single ASOs (Single ASO) or by a combination of two of them (Combined ASOs).

The figure showed the means (sd) obtained with 4 single ASOs (namely ASO-29391, ASO-507, ASO-29439, ASO-15444) and their combinations. The best two combinations were: ASO-29391+ASO-507 and ASO-29391+ASO-15444. Note that all these tests were performed altogether under the same experimental conditions for a better standardization (HEK293T/ACE2 cells infected by SARS-CoV-2 at MOI: 0.2). The inventors expressed the means per group Single ASO vs Combined ASOs and performed a non-parametric statistical test for comparison of the two groups (p<0.05).

FIG. 4: Prophylactic treatment against a SARS-CoV-2 infection with ASO-29391

Flash treatment by nose instillation (75% dose) and intra-peritoneal injection (25% dose). The Golden Hamsters were first treated with ASO-29391, then, 2 hours later, were infected by the nose with a virus load of 5000 PFU. N-CoV qPCR was carried out 24 hours post infection to measure viral genome copies in treated group and in the placebo group. The quantity of viral RNA copies (N-CoV) reached 42% reduction in the ASO treated group in comparison with the placebo group.

FIG. 5: Animals treated with ASO-29391 after a SARS-CoV-2 infection lose weight slower than placebo group

Time-series of weight changes of the animals (N=8 Treated vs 8 Placebo) between Day 1 and Day 4.

FIG. 6: Animals treated with ASO-29391 after a SARS-CoV-2 infection present a food intake higher than their control counterparts Time-series of the food intake between Day 1 and Day 4 in the treated group (N=8) and placebo group (N=8).

FIG. 7: Viral ORF1ab RNA relative quantification on Day 4 in the nasal mucosa of animals treated with ASO-29391 versus placebo group.

The relative viral expression of ORF1ab was measured by RT-qPCR normalized by a reference gene UEB2D2. The relative quantity of ORF1ab RNA reached a significant 35% reduction in the ASO treated group in comparison with the placebo group (p<0.05).

FIG. 8: Comparison of the efficiency of ASO-29391 with different chemistries: PS, 2′OME, and LNA and a siRNA having targeting the same viral sequence.

Dunnett's T3 multiple comparisons test and Welch's t-test. *p<0.05, **p<0.01, ****p<0,0001. RLU: Relative Luminescence units, RFU Relative fluorescence units. Luciferase assay subtract Promega was used for the luciferase reaction. Red fluorescence was read for transfection control at 556/9 nm excitation 584/20 nm emission in Tecan plate reader in Costar white flat bottom 96 well plates.

FIG. 9: Comparison of the efficiency of ASO-29391 with PS chemistry (RNAse H1 dependent cleaving activity) and siRNA having the same sequence but with different cleaving activity pathway pathway (miRNRA maturation pathway) at increasing doses.

RLU: Relative Luminescence units, RFU Relative fluorescence units. Luciferase assay subtract Promega was used for the luciferase reaction. Red fluorescence was read for transfection control at 556/9 nm excitation 584/20 nm emission in Tecan plate reader in Costar white flat bottom 96 well plates.

FIG. 10: The combination of RNAse H1 dependent ASO-29391 (PS or LNA) with steric blocking ASO-29391 (MPO) increases its antiviral effect against SARS-CoV-2 infected cells

Significant differences (*p<0.05; **p<0.01) were determined by one-way Anova. Raw TCID50 results were transformed in PFU/ml.

FIG. 11: The combination of RNAse H1 dependent ASO-29391 (PS or LNA-data pooled) with steric blocking ASO-29391 (MPO) increases 2 times its antiviral effect against SARS-CoV-2 infected cells. By using the mixture 50:50 of the two types of ASOs a synergic effect is observed.

Significant differences (*p<0.05; ****p<0.0001) were determined by one-tailed unpaired t-test.

FIG. 12: Boosted transfection induces a strong decrease in fluorescence in SARS-CoV-2/Zsgreen infected VeroE6/TMPRSS2 cells with both RNase H1 dependent ASOs (PS and LNA; *p<0.05) while steric blocking oligonucleotides (MPO and PNA) fail to induce any decrease. Fluorescence are normalized by DAPI.

FIG. 13: Mixtures of RNAse H1 dependent and steric blocking oligonucleotides act synergically to decrease SARS-CoV-2/Zsgreen fluorescence in VeroE6/TMPRSS2 cells. Fluorescence are normalized by DAPI. Significant differences were determined by one-tailed unpaired t-test *p<0.05.

### DETAILED DESCRIPTION OF THE INVENTION

The present invention provides new antisense oligonucleotides, pharmaceutical compositions comprising them, and their use to treat or prevent coronavirus infection, in particular infection by SARS-CoV-2. The inventors identified key viral genome sequence involved early in the replication process: 5′UTR, ORF1a, ORF1b, gene N and 3′UTR, especially the N gene region, being the most transcribed and expressed gene of the viral genome. The N-ORF10-3′UTR region was also of interest as a target in order to block the transcription and regulation process of the replication, with stem loops and domains that are shown to interact with the 5′UTR-ORF1ab region.

More specifically, the inventors have identified sequences, especially in the N, 5′UTR and ORF1ab regions, that prove to be great targets to inhibit the replication of SARS-CoV-2. More specifically, the inventors have identified highly conserved sequences among SARS-CoV-2 variants, in the N region and ORF1ab nsp12 region, with a reduced likelihood of showing evolving mutations in the future that could lead to potential resistance. Indeed, ASO of SEQ ID NO: 1 and 2 (called respectively ASO-29391 and ASO-15444) show a perfect matching with their targeted sequence in many variants of the SARS-CoV-2 genome and in additional multiple secondary targets in the SARS-CoV-2 genome (both positive and negative strands, both useful in the replication process).

Surprisingly, the inventors found out that the targeting of these sequences with ASOs can lead to an inhibition of the replication of SARS-CoV-2 virus up to 94-95% in vitro for the best ASO, namely ASO-29391.

The present invention results from a different strategy, where targeted sequences are not only chosen for their importance in the machinery of the virus, but also because of the number of hits on the targeted genome. Accordingly, a great candidate for the inhibition of the replication is an ASO showing a great number of hits on the targeted genome.

Indeed, and without being bound by theory, the ASO of the invention presents an important number of different hit sites on the targeted genome, for instance 88 hits for the ASO of SEQ ID NO: 1 (ASO-29391) can be found on the targeted genome and 28 for the ASO of SEQ ID NO: 2 (ASO-15444). This level of occurrence in the targeted genome for an ASO target site within SARS-CoV-2 genome is undisclosed. Targeting SARS-CoV-2 highly conserved sequence with multiple secondary target sites is of particular interest in successfully inhibiting the replication of the virus, and represents a novel strategy in the global ongoing fight against SARS-CoV-2. In addition, it has been checked that there is no significant off target in the human genome (No plus/minus blast over 15 nucleotides without mismatch).

To our knowledge, none of the cited documents disclose both highly conserved sequence and multiple secondary target sites for an ASO (on the viral genome) on both positive and negative strands.

### Definitions

“SARS” refers to severe acute respiratory syndrome.

“SARS-CoV” refers to severe acute respiratory syndrome-associated coronavirus. “SARS-CoV-1” refers to severe acute respiratory syndrome-associated coronavirus 1. “SARS-CoV-2” refers to severe acute respiratory syndrome-associated coronavirus 2 emerging officially in Wuhan late 2019. “MERS-CoV” refers to Middle Eastern respiratory syndrome-associated coronavirus.

SARS-CoV-2 genome refers to the Severe Acute Respiratory Syndrome Coronavirus 2 isolate Wuhan-Hu-1, complete genome (Wu et al. 2020, Nature 579, 265-269. doi: 10.1038/s41586-020-2008-3) and the variants of concern and sublineages (VOC defined by WHO: alpha, Beta, Delta, Mu, Omicron, and now Omicron sublineages). It is disclosed in the Genbank database under NC_045512.2. SARS-CoV-2 genome comprises 14 regions consisting of 5′UTR, ORF1a, FSE region, ORF1a/b, ORF1b, S, ORF3a, E, M, ORF6, ORF7a, ORF7b, ORF8, N, ORF10 and 3′ UTR and encodes 16 non-structural proteins (nsp) consisting of nsp1, nsp2, nsp3, nsp4, nsp5, nsp6, nsp7, nsp8, nsp9, nsp10, nsp11, nsp 12, nsp13, nsp 14, nsp15 or nsp16, a Spike(S) protein, an Envelope (E) protein, a Membrane (M) glycoprotein, and a Nucleocapsid (N) protein. “COVID-19” refers to the disease caused by SARS-CoV-2. Symptoms include non-exhaustively coughing, sore throat, runny nose, sneezing, anosmia, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, hemoptysis, conjunctival congestion, sputum production, chest tightness, and palpitations, fever, biochemical disorders, cytokine storm and other signals.

As used herein, the terms “essentially consists in” refer to at least 75%, 85%, 90%, 95%, 96%, 97%, 98%, 99% or 100% identity to a sequence. Alternatively, the terms refer to a sequence comprising 1, 2, 3, 4 or 5 mutations (addition, deletion and/or substitution). In a particular aspect, it refers to a sequence comprising/including/having 1, 2 or 3 nucleotide single mutations at one or both ends of the sequence. In a very particular aspect, it refers to a sequence comprising/including/having 1) the addition of 1, 2 or 3 nucleotides at one or both ends of the sequence; 2) the deletion of 1, 2 or 3 nucleotides at one or both ends of the sequence; or 3) the substitution of 1, 2 or 3 nucleotides at one or both ends of the sequence.

The term “target” means that the ASO is complementary or capable of hybridizing a particular sequence or site. “Hybridize” and “hybridization” refer to the pairing of complementary (including partially complementary) nucleic acid strands. When one nucleic acid is said to “hybridize” to another nucleic acid, it means that there is some complementarity between the two nucleic acids or that the two nucleic acids form a hybrid under high or low stringency conditions.

The term “complementary” refers to a nucleotide (e.g., RNA or DNA) or a sequence of nucleotides capable of base pairing with a complementary nucleotide or sequence of nucleotides. As described herein and commonly known in the art, the complementary (matching) nucleotide of adenosine is thymidine (in DNA) or uracil (in RNA) and the complementary (matching) nucleotide of guanidine is cytosine. Thus, a complement may include a sequence of nucleotides that base pair with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Percentage of sequence identity is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Identity can be measured using a BLAST or BLAST 2.0 sequence comparison algorithms with default parameters described below, or by manual alignment and visual inspection (e.g., http://www.ncbi.nlm.nih.gov/BLAST/or the like).

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

As used herein, the term “effective amount” or “therapeutically effective amount”, means the quantity of the pharmaceutical composition, kit, product and combined preparation of the invention which prevents, delays, removes or reduces the deleterious effects of infection by a coronavirus in mammals, including humans, alone or in combination with the other active ingredients of the pharmaceutical composition, kit, product or combined preparation. It is understood that the administered dose may be adapted by those skilled in the art according to the patient, the pathology, the mode of administration, etc. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “infection by a coronavirus” designates any infection or disease caused by any coronavirus.

“Patient” or “subject” refers to a living organism. Non-limiting examples include humans, other mammals, bovines, rats, mice, dogs, monkeys, goat, sheep, cows, and other non-mammalian animals. In a preferred aspect, a patient is human.

As used herein, the terms “treating,” “treatment” or “therapy” with reference to a coronavirus infections refer to reducing or eliminating viral load, and/or improving, alleviating or ameliorating one or more symptoms of the infection such as coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, hemoptysis, conjunctival congestion, sputum production, chest tightness, and palpitations, fever and/or cytokine storm, diminishing the extent of a disease, stabilizing (i.e., not worsening) the state of disease, preventing a disease's transmission or spread, delaying or slowing disease progression, diminishing the reoccurrence of disease, and reaching remission, whether partial or total and whether detectable or undetectable.

“Treating” or “treatment” as used herein (and as well-understood in the art) also broadly includes any approach for obtaining beneficial or desired results in a subject's condition, including clinical results. Beneficial or desired clinical results can include, but are not limited to, alleviation or amelioration of one or more symptoms or conditions. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

As used herein, the terms “prevent” or “preventing” with reference to a coronavirus infections refer to precluding an infection from developing in a subject exposed to a coronavirus and/or avoiding the development of one or more symptoms of an infection such as coughing, sore throat, runny nose, sneezing, headache, fever, shortness of breath, myalgia, abdominal pain, fatigue, difficulty breathing, persistent chest pain or pressure, difficulty waking, loss of smell and taste, muscle or joint pain, chills, nausea or vomiting, nasal congestion, diarrhea, hemoptysis, conjunctival congestion, sputum production, chest tightness, and palpitations, fever and/or cytokine storm. “Prevention” may occur when the viral load is never allowed to exceed beyond a threshold level, for instance a threshold level at which point the subject begins to feel sick or exhibit symptoms. By prevent” or “preventing”, it could refer to a prophylactic treatment. “Prevention” may also, in some embodiments, refer to the prevention of a subsequent infection once an initial infection has been treated or cured.

The terms “kit”, “product” or “combined preparation”, as used herein, are interchangeable and define especially a “kit-of-parts” in the sense that the combination partners (a) and (b), as defined above can be dosed independently or by use of different fixed combinations with distinct amounts of the combination partners (a) and (b), i.e. simultaneously or at different time points. The components of the kit-of-parts can then, e.g., be administered simultaneously or chronologically staggered, that is at different time points and with equal or different time intervals for any part of the kit-of-parts. The ratio of the total amounts of the combination partner (a) to the combination partner (b), to be administered in the combined preparation can be varied. The combination partners (a) and (b) can be administered by the same route or by different routes. In particular, these terms refer to a product or kit containing the combination partner (a) and the combination partner (b) for simultaneous, separate or sequential use.

The term “administering” means intranasal administration, inhalation administration, oral administration, administration as a suppository, topical contact, intravenous, parenteral, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery may include the use of lipid nanoparticles, viral vectors, aerosols, liposomal formulations, intravenous infusion, transdermal patches, and the like. Optionally, administering does not include administration of any active agent other than the ASO. In some aspects, administration is intranasal. In other aspects, administration is intravenous. In still other aspects, administration is by inhalation or nebulization, especially mouth inhalation or nebulization.

### Antisense Oligonucleotide

The present invention relates to an antisense oligonucleotide targeting a coronavirus genome or to an ASO inhibiting the replication of the coronavirus.

As used herein, an “ASO” refers to a modified single-stranded oligonucleotide comprising at least one region which is complementary to a target nucleic acid. ASOs are designed and commonly used to modulate the expression of their target nucleic acid, notably to knock down their target. The precise targeting of a specific nucleic acid, on which the selectivity of a knockdown strategy depends, relates to a balance between oligonucleotide length and complementarity rate toward the defined target. In addition, chemical modifications are generally required to confer an improvement in single-stranded oligonucleotide stability, especially towards digestion by nucleases. Indeed, unmodified single-stranded oligonucleotides are too instable to use in cells. Notably, it is well known by the one skilled in the art that the nuclease resistance can be dramatically improved by modifying internucleotide linkage, e.g., by substituting phosphodiester bonds by phosphorothioate (PS) linkage. Furthermore, other chemical modifications can improve potency and selectivity of ASO by increasing binding affinity of ASOs for their target.

The ASO according to the invention comprises, essentially consists in or consists in a sequence set forth in SEQ ID NO: 1. Preferably, the ASO according to the invention comprises or consists in a sequence of SEQ ID NO: 1. Indeed, ASO-29391 has shown the best efficiency to prevent and treat coronavirus infection as shown in the examples and especially as summarized in Table 3. Its target is in N gene and it presents 88 additional secondary targets in the SARS-CoV-2 genome. Its target is highly conserved among the coronavirus variants as shown in Table 2. For instance, the ASO of the invention could be a longer molecule including the sequence of SEQ ID NO: 1, such as an ASO comprising the sequence of SEQ ID NO: 1 flanked at its 5′ and/or 3′ ends by 1-5 additional nucleotides (e.g., 1, 2, 3, 4 or 5 additional nucleotides). Alternatively, the ASO of the invention could also be a shorter molecule including a sequence of SEQ ID NO: 1 with the deletion of 1-5 nucleotides (e.g., 1, 2, 3, 4 or 5 deleted nucleotides), the deletion being preferably done at the ends of the sequence of SEQ ID NO: 1. Such a shorter ASO would include at least 15, 16, 17, 18 or 19 consecutive nucleotides of SEQ ID NO: 1. In another aspect, the ASO of the invention could include the sequence of SEQ ID NO: 1 with a substitution of 1-5 nucleotides, thereby introducing 1-5 mismatches (e.g., 1, 2, 3, 4 or 5 mismatched nucleotides, preferably not consecutive nucleotides).

In a particular aspect, the present invention relates to a pharmaceutical composition comprising the ASO comprising, essentially consisting in or consisting in a sequence set forth in SEQ ID NO: 1 and one or several other ASOs targeting SARS-CoV-2 genome; or the combined use or use in combination of with the ASO comprising, essentially consisting in or consisting in a sequence set forth in SEQ ID NO: 1 and one or several other ASOs targeting SARS-CoV-2 genome. In a particular aspect, the ASO comprising, essentially consisting in or consisting in a sequence set forth in SEQ ID NO: 1 is with modification(s), optionally is any ASO as disclosed in Table A (SEQ ID NOs: 24 and 45-103) or a combination thereof.

Accordingly, other ASOs are complementary to SARS-CoV-2 genome. Especially, they may have a target site in 5′ UTR, ORF1a, ORF1b, S, ORF3a, E, M, ORF6, ORF7a, ORF7b, ORF8, N, ORF10, or 3′ UTR regions, more preferably in 5′UTR, ORF1ab-nsp1, ORF1ab-nsp12, ORF1ab-FSE, or N. Optionally, the other ASOs can be any ASO already available in the art, for instance any of the ASOs disclosed in Hedge et al, 2021, supra; Quemener and Galibert, 2021, supra; Zhu et al., 2022, supra; Vora et al., 2022, supra; Li et al, 2021, supra; Zhang et al, 2021, supra; Lulla et al, 2021, supra; Dauksaite et al, 2022, supra; Dhorne-Pollet et al, 2022, supra; WO2022/031410; WO2021/207641 and WO2021/195025, the disclosure of which being incorporated herein by reference.

In a particular aspect, the present invention relates to a pharmaceutical composition comprising the ASO comprising, essentially consisting in or consisting in a sequence set forth in SEQ ID NO: 1 and one or several ASOs targeting either the N gene or the ORF1ab, especially ORF1ab-nsp1 or ORF1ab-nsp12. In a particular aspect, the ASO comprising, essentially consisting in or consisting in a sequence set forth in SEQ ID NO: 1 is with modification(s), optionally is any ASO as disclosed in Table A (SEQ ID NOs: 24, 45-103 and 105, optionally 24 and 45-103) or a combination thereof. Optionally, the ASO is any ASO as disclosed in Table A under SEQ ID NOs: 24, 45-101 and 105, or under SEQ ID NOs: 24, 45-57, 80-101 and 105. In a very specific aspect, the ASO is any ASO as disclosed in Table A under SEQ ID NOs: 24, 48 and 105.

The inventors have identified other ASOs of interest, that can be used alone or in combination for preventing or treating coronavirus infection, preferably in combination with the ASO comprising, essentially consisting in or consisting in a sequence set forth in SEQ ID NO: 1, in particular an ASO with modification(s), optionally any ASO as disclosed in Table A (SEQ ID NOs: 24, 45-103 and 105, optionally 24 and 45-103) or a combination thereof. Optionally, the ASO is any ASO as disclosed in Table A under SEQ ID NOs: 24, 45-101 and 105, or under SEQ ID NOs: 24, 45-57, 80-101 and 105. In a very specific aspect, the ASO is any ASO as disclosed in Table A under SEQ ID NOs: 24, 48 and 105. In particular, the present disclosure relates to an ASO comprising, essentially consisting in or consisting in any of the sequences of SEQ ID NO: 2-21, preferably any of the sequences of SEQ ID NO: 2-6, still more preferably a sequence of SEQ ID NO: 2. ASO of SEQ ID NO: 2-6 are respectively called ASO-15444, ASO-29446, ASO-507, ASO-60 and ASO-13503. They target N gene, ORF1ab, especially ORF1ab nsp1, ORF1ab nsp12 or ORF1ab FSE, or 5′ UTR region. Their targets are disclosed in Table 1. ASO-15444 is of special interest because, beside the targeting of ORF1ab nsp12, it presents 28 additional secondary targets in the SARS-CoV-2 genome and it also highly conserved among the coronavirus variants as shown in Table 2.

In a particular aspect, the present invention relates to a pharmaceutical composition or a combined preparation comprising the ASO comprising, essentially consisting in or consisting in a sequence set forth in SEQ ID NO: 1 and one or several ASOs comprising, essentially consisting in or consisting in any of the sequences of SEQ ID NO: 2-21, preferably any of the sequences of SEQ ID NO: 2-6, still more preferably a sequence of SEQ ID NO: 2. In a particular aspect, the ASO comprising, essentially consisting in or consisting in a sequence set forth in SEQ ID NO: 1 is with modification(s), optionally is any ASO as disclosed in Table A (SEQ ID NOs: 24, 45-103 and 105, optionally 24 and 45-103) or a combination thereof. Optionally, the ASO is any ASO as disclosed in Table A under SEQ ID NOs: 24, 45-101 and 105, or under SEQ ID NOs: 24, 45-57, 80-101 and 105. In a very specific aspect, the ASO is any ASO as disclosed in Table A under SEQ ID NOs: 24, 48 and 105.

In a preferred aspect, the other ASO nucleotide sequence is complementary to 5′UTR, ORF1ab, or N region. More preferably, the ASO nucleotide sequence is complementary to 5′UTR, ORF1ab-nsp1, ORF1ab-nsp12, ORF1ab-FSE, or N. More preferably, the ASO nucleotide sequence is complementary to N region.

More specifically, the other ASO nucleotide sequence can be complementary to 5′UTR and it comprises, essentially consists in or consists in any of the sequences of SEQ ID NOs: 5 and 7-10, preferably SEQ ID NO: 5.

More specifically, the other ASO nucleotide sequence can be complementary to ORF1ab region of SARS-CoV-2 genome and it comprises, essentially consists in or consists in any of the sequences of SEQ ID NOs: 2, 4 and 11-18, preferably any of the sequences of SEQ ID NOs: 2 and 4. Optionally, the ASO nucleotide sequence can be complementary to ORF1ab-nsp1 region of SARS-CoV-2 genome and it comprises, essentially consists in or consists in a sequence of SEQ ID NO: 4. Optionally, the ASO nucleotide sequence can be complementary to ORF1ab-nsp12 region of SARS-CoV-2 genome and it comprises, essentially consists in or consists in a sequence of SEQ ID NO: 2.

More specifically, the other ASO nucleotide sequence can be complementary to N region of SARS-CoV-2 genome and it comprises, essentially consists in or consists in any of the sequences of SEQ ID NOs: 1, 3 and 19, preferably any of the sequences of SEQ ID NOs: 1 and 3.

More specifically, the other ASO nucleotide sequence can be complementary to ORF10 region of SARS-CoV-2 genome and it comprises, essentially consists in or consists in the sequence of SEQ ID NO: 20. More specifically, the other ASO nucleotide sequence can be complementary to 3′UTR region of SARS-CoV-2 genome and it comprises, essentially consists in or consists in the sequence of SEQ ID NO: 21.

In a particular aspect, the present invention relates to a pharmaceutical composition or a combined preparation comprising at least 2 ASOs having the same target in the SARS-CoV-2 genome and presenting different chemical modifications leading to two different mechanisms of action, namely at least one ASO for cleaving by RNAse H1 and at least one ASO for steric blocking (e.g., PMO or PNA). Such a combination provides the advantage of avoiding RNAse H1 saturation with two ASOs at high dose and inducing the same cleaving mechanism. Optionally, the pharmaceutical composition comprises one ASO comprising phosphorothioate (PS) linkage modification and/or nucleotide modifications selected from the group consisting of LNA modifications and 2′-O-Me modifications and one ASO in which all nucleotides are phosphorodiamidate morpholino oligomer (PMO) or peptide nucleic acid oligomer (PNA).

Optionally, the at least 2 ASOs comprise, essentially consist in or consist in a sequence set forth in SEQ ID NO: 1. Alternatively, the at least 2 ASOs comprise, essentially consist in or consist in a sequence set forth in SEQ ID NO: 3.

In another particular aspect, the present invention relates to a pharmaceutical composition or a combined preparation comprising at least 2 ASOs having a different target in the SARS-CoV-2 genome, optionally two target sites close to each other, especially target sites located at the end of the SARS-CoV-2 genome such as in the N gene and 3′ UTR region. Optionally, the at least 2 ASOs may present different chemical modifications leading to two different mechanisms of action, namely at least one ASO for cleaving by RNAse H1 and at least one ASO for steric blocking (e.g., PMO or PNA). Such a combination provides the advantage of avoiding RNAse H1 saturation with two ASOs at high dose and inducing the same cleaving mechanism.

In very particular aspects, the present invention relates to a pharmaceutical composition or a combined preparation comprising:
the ASO comprising, essentially consisting in or consisting in a sequence set forth in SEQ ID NO: 1 and the ASO comprising, essentially consisting in or consisting in a sequence set forth in SEQ ID NO: 2; or
the ASO comprising, essentially consisting in or consisting in a sequence set forth in SEQ ID NO: 1 and the ASO comprising, essentially consisting in or consisting in a sequence set forth in SEQ ID NO: 3; or
the ASO comprising, essentially consisting in or consisting in a sequence set forth in SEQ ID NO: 1 and the ASO comprising, essentially consisting in or consisting in a sequence set forth in SEQ ID NO: 4; or
the ASO comprising, essentially consisting in or consisting in a sequence set forth in SEQ ID NO: 3 and the ASO comprising, essentially consisting in or consisting in a sequence set forth in SEQ ID NO: 4.

In very particular aspects, the present invention relates to a pharmaceutical composition or a combined preparation comprising:
the ASO comprising, essentially consisting in or consisting in a sequence set forth in SEQ ID NO: 1 and the ASO comprising, essentially consisting in or consisting in a sequence set forth in SEQ ID NO: 3; or
the ASO comprising, essentially consisting in or consisting in a sequence set forth in SEQ ID NO: 1 and the ASO comprising, essentially consisting in or consisting in a sequence set forth in SEQ ID NO: 19; or
the ASO comprising, essentially consisting in or consisting in a sequence set forth in SEQ ID NO: 3 and the ASO comprising, essentially consisting in or consisting in a sequence set forth in SEQ ID NO: 19.

In particular aspect, the ASO comprising, essentially consisting in or consisting in a sequence set forth in SEQ ID NO: 1 is with modification(s), optionally is any ASO as disclosed in Table A (SEQ ID NOs: 24 and 45-103) or a combination thereof.

In a particular aspect, the pharmaceutical composition comprises one ASO selected from the group consisting of SEQ ID NOs 24, 45-101 and 105, and one ASO of selected from the group consisting of SEQ ID NOs 102 and 103. In a more specific aspect, the pharmaceutical composition comprises one ASO selected from the group consisting of SEQ ID NOs 24, 48 and 105, and one ASO of SEQ ID NO: 102 or 103. Optionally, the ASO may comprise, essentially consist in or consist in one of the disclosed SEQ ID NOs and further comprise 1, 2 or 3 mismatched nucleotides in comparison to the targeted sequence.

Preferably, the ASO according to the invention comprises a nucleotide sequence of 16 to 30 nucleotides in length. Preferably, the length of the ASO sequence is 18 to 28 nucleotides, more preferably 20 to 26, 20 to 24, 20 to 22, 19 to 21 or 20 nucleotides in length.

The ASO according to the invention is a single-stranded oligonucleotide comprising deoxyribonucleotides and/or ribonucleotides. In a first aspect, the ASO according to the invention comprises ribonucleotides and deoxyribonucleotides, i.e. DNA or DNA-like nucleotides. In a second aspect, the ASO according to the invention comprises only deoxyribonucleotides, i.e. DNA or DNA-like nucleotides. In a third aspect, the ASO according to the invention comprises only ribonucleotides, i.e. RNA or RNA-like nucleotides. Depending on their composition in nucleotides, i.e. ribonucleotides and deoxyribonucleotides, deoxyribonucleotides or ribonucleotides, the ASO may inhibit the expression of its target nucleic acid via different mechanisms.

In a preferred aspect, the ASO acts via RNase H1 mediated degradation. RNase H1 is a cellular enzyme which recognizes the duplex between DNA and RNA, and enzymatically cleaves the RNA molecule. Thus, ASO comprises a region that comprises DNA or DNA-like nucleotides complementary to the targeted nucleic acid and is responsible for inducing RNAse H1 recruitment that leads to subsequent target nucleic acid cleavage.

Alternatively, the ASO according to the invention may act through steric hindrance (Hagedorn, P. H. et al. Locked nucleic acid: modality, diversity, and drug discovery. Drug Discovery Today 23, 101-114 (2018) doi: 10.1016/j.drudis.2017.09.018.).

The ASO according to the invention comprises chemical modifications that confer an improved stability of single-stranded oligonucleotides, for instance modifications relative to internucleotide linkages. In a preferred aspect, the ASO according to the invention comprises phosphorothioate (PS) linkages in place of some phosphodiester bonds. The phosphorothioate linkages are preferably localized at the ends of the ASO. For instance, the PS linkages can be present for at least the 3 to 5 terminal 5′ and/or 3′ nucleotides, preferably both at the 5′ and 3′ ends. Optionally, the ASO according to the invention comprises at least 10, 11, 12, 13, 14, 15, 16, 17, 18, 19, 20 or 21 phosphorothioate linkages. In a particular aspect, all the internucleotide linkage of ASO are phosphorothioate linkages.

In a particular aspect, alternatively or in combination with the phosphorothioate (PS) linkages, the ASO according to the invention comprises nucleotide modifications. Such nucleotide modifications can be selected among, but are not limited to, the addition on ribose of group such as 2′-O-methyl(2′-O-Me), 2′-O-methoxyethyl(2′-O-MOE), 2′-fluoro (2′-F), the introduction of a methyl (CH₃) group at the 5-carbon (5-M) of the pyrimidine ring of a cytosine or uracil nucleotide, the introduction of methylene bridge between the 2′ and 4′ positions of the ribose which define the “locked” nucleic acids (LNAs), the introduction of an ethylene bridge with a cyclopropane ring between the 3′ and 5′ positions of the ribose which define the tricyclo-DNA (tcDNA) or the introduction of constrained ethyl (cEt) bridged nucleic acid (BNA).

Besides, phosphorodiamidate morpholino oligomers (PMOs) represent another modification that enhances metabolic stability and affinity for ribonucleotides by replacing the sugar and backbone with a morpholino ring system.

All these modifications are well known to the one skilled in the art and also contribute to enhance the oligonucleotide stability (see Watts et al. J Pathol. 2012 January; 226 (2): 365-379; Seth et al. J Clin Invest. 2019; 129 (3): 915-925).

Optionally, the ASO according to the invention comprises one or more additional compound at any end of the ASO, to increase the intracellular delivery of said compounds by gymnosis. Preferably, the ASO according to the invention comprises one or more compound at both ends. More preferably, the ASO comprises compounds at one end. In a particular aspect, the compounds are selected from several amino acids Lys, Arg and/or Cys or any cholesterol-derived compound.

In particular aspect, the ASO according to the invention comprises 2′-O-Me, 2′-O-MOE, 2′-F, 5-M, LNA, tcDNA, cET and/or PMO modified nucleotides in the nucleotide sequence, preferably 2′-O-Me, 2′-O-MOE, 2′-F, 5-M, and/or LNA modified nucleotides, more preferably 2′-O-Me, 2′-O-MOE, 5-M, and/or LNA modified nucleotides and combinations thereof.

Optionally, the ASO according to the invention comprises at least 1, 2, 3, 4, 5, 6, 7, 8, 9, 10, 11, 12, 13, 14, 15, 16, 17, 18, 19, 20 or 21 2′-O-Me modified nucleotides; and/or at least 1, 2, 3, 4, 5, 6, 7, 8, 9, 10, 11, 12, 13, 14, 15, 16, 17, 18, 19, 20 or 21 2′-O-MOE modified nucleotides; and/or at least 1, 2, 3, 4, 5, 6, 7, 8, 9, 10, 11, 12, 13, 14, 15, 16, 17, 18, 19, 20 or 21 5-M modified nucleotides; and/or at least 1, 2, 3, 4, 5, 6, 7, 8, 9, 10, 11, 12, 13, 14, 15, 16, 17, 18, 19, 20 or 21 LNA modified nucleotides. Optionally, the ASO according to the invention comprises at least 1, 2, 3, 4, 5, 6, 7, 8, 9, 10, 11, 12, 13, 14, 15, 16, 17, 18, 19, 20 or 21 5-M modified nucleotides and at least 1, 2, 3, 4, 5, 6, 7, 8, 9, 10, 11, 12, 13, 14, 15, 16, 17, 18, 19, 20 or 21 LNA modified nucleotides. Optionally, the ASO according to the invention comprises at least 1, 2, 3, 4, 5, 6, 7, 8, 9, 10, 11, 12, 13, 14, 15, 16, 17, 18, 19, 20 or 21 5-M modified nucleotides, at least 1, 2, 3, 4, 5, 6, 7, 8, 9, 10, 11, 12, 13, 14, 15, 16, 17, 18, 19, 20 or 21 2′-O-Me modified nucleotides. Optionally, the ASO according to the invention comprises at least 1, 2, 3, 4, 5, 6, 7, 8, 9, 10, 11, 12, 13, 14, 15, 16, 17, 18, 19, 20 or 21 5-M modified nucleotides, at least 1, 2, 3, 4, 5, 6, 7, 8, 9, 10, 11, 12, 13, 14, 15, 16, 17, 18, 19, 20 or 21 2′-O-MOE modified nucleotides.

Optionally, all nucleotides of the ASO are 2′-O-MOE. In addition, the 4-5 nucleotides at each end of the ASO present phosphorothioate linkages. Optionally, all nucleotide linkages are phosphorothioate linkages. In addition, some or all cytosines or uracils of the ASO are modified and are 5-methylated.

In a very specific aspect, the ASO comprises, essentially consists in or consists in a sequence set forth in SEQ ID NO: 1 and all nucleotides are 2′-O-MOE nucleotides. Optionally, all nucleotide linkages are phosphorothioate linkages. Alternatively, the 4-5 nucleotides at each end of the ASO present phosphorothioate linkages. Optionally, the cytosines are 5-methyl cytosines. The same configuration can be applied to any ASO comprising, essentially consisting in or consisting in any of the sequences set forth in SEQ ID NO: 2-21.

Optionally, all nucleotides of the ASO are 2′-O-Me. In addition, the 4-5 nucleotides at each ends of the ASO present phosphorothioate linkages. Optionally, all nucleotide linkages are phosphorothioate linkages. In addition, some or all cytosines or uracils of the ASO are modified and are 5-methylated. In a very specific aspect, the ASO comprises, essentially consists in or consists in a sequence set forth in SEQ ID NO: 1 and all nucleotides are 2′-O-Me nucleotides. Optionally, all nucleotide linkages are phosphorothioate linkages. Alternatively, the 4-5 nucleotides at each end of the ASO present phosphorothioate linkages. Optionally, the cytosines are 5′methyl cytosines. The same configuration can be applied to any ASO comprising, essentially consisting in or consisting in any of the sequences set forth in SEQ ID NO: 2-21.

Optionally, the 3-5 nucleotides at each end of the ASO are modified nucleotides, especially LNA nucleotides. In addition, the 4-5 nucleotides at each end of the ASO present phosphorothioate linkages. Optionally, all nucleotide linkages are phosphorothioate linkages. Optionally, the ASO may further comprise modified nucleotides such as 2′-O-Me or 2′-O-MOE nucleotides, preferably 2′-O-Me nucleotides. In addition, some or all cytosines or uracils of the ASO are modified and are 5-methylated.

In a very specific aspect, the ASO comprises, essentially consists in or consists in a sequence set forth in SEQ ID NO: 1 and the 3-5 nucleotides at each end of the ASO are LNA nucleotides. Optionally, the other nucleotides are 2′-O-Me nucleotides. Optionally, all nucleotide linkages are phosphorothioate linkages. Alternatively, the 4-5 nucleotides at each end of the ASO present phosphorothioate linkages. Optionally, the cytosines are 5-methyl cytosines. The same configuration can be applied to any ASO comprising, essentially consisting in or consisting in any of the sequences set forth in SEQ ID NO: 2-21.

Optionally, the 3-5 nucleotides at each end of the ASO are modified nucleotides, especially 2′-O-Me nucleotides. In addition, the 4-5 nucleotides at each end of the ASO present phosphorothioate linkages. Optionally, all nucleotide linkages are phosphorothioate linkages. In addition, some or all cytosines or uracils of the ASO are modified and are 5-methylated.

In a very specific aspect, the ASO comprises, essentially consists in or consists in a sequence set forth in SEQ ID NO: 1 and the 3-5 nucleotides at each end of the ASO are 2′-O-Me nucleotides. Optionally, all nucleotide linkages are phosphorothioate linkages. Alternatively, the 4-5 nucleotides at each end of the ASO present phosphorothioate linkages. Optionally, the cytosines are 5-methyl cytosines. The same configuration can be applied to any ASO comprising, essentially consisting in or consisting in any of the sequences set forth in SEQ ID NO: 2-21.

Optionally, the ASO comprising some LNA modified nucleotides, with one LNA modified nucleotide at each end and at least other 2-3 other LNA modified nucleotides within the ASO.

Optionally, all nucleotides of the ASO are phosphorodiamidate morpholino oligomer (PMO). Alternatively, all nucleotides of the ASO are peptide nucleic acid oligomer (PNA). Alternatively, all nucleotides of the ASO are peptide-conjugated phosphoroamidate morpholino oligomer (PPMO).

In a very specific aspect, the ASO comprises, essentially consists in or consists in a sequence set forth in SEQ ID NO: 1 and all nucleotides of the ASO are phosphorodiamidate morpholino oligomer (PMO) or peptide nucleic acid (PNA). The same configuration can be applied to any ASO comprising, essentially consisting in or consisting in any of the sequences set forth in SEQ ID NO: 2-21.

For instance, please find in the following Table A a non-exhaustive illustration of ASOs having a sequence of SEQ ID NO: 1.

TABLE A
ASO of SEQ ID NO: 1 with various modifications

| SEQ | |
|---|---|
| ID NO | ASO-29391 |
| 1 | A-G-T-T-T-C-A-T-C-A-G-C-C-T-T-C-T-T-C-T |
| 24 | As-Gs-Ts-Ts-Ts-Cs-As-Ts-Cs-As-Gs-Cs-Cs-Ts-Ts-Cs-Ts-Ts-Cs-T |
| 45 | As-Gs-Ts-Ts-Ts-mCs-As-Ts-mCs-As-Gs-mCs-mCs-Ts-Ts-mCs-Ts-Ts-mCs-T |
| 46 | IA-IG-IT-IT-T-C-A-T-C-A-G-C-C-T-T-C-IT-IT-IC-IT |
| 47 | IA-IG-IT-IT-T-mC-A-T-mC-A-G-mC-mC-T-T-mC-IT-IT-mIC-IT |
| 48 | IAs-IGs-ITs-ITs-Ts-Cs-As-Ts-Cs-As-Gs-Cs-Cs-Ts-Ts-Cs-ITs-ITs-ICs-IT |
| 49 | IAs-IGs-ITs-ITs-Ts-mCs-As-Ts-mCs-As-Gs-mCs-mCs-Ts-Ts-mCs-ITs-ITs-mICs-IT |
| 50 | IA-IG-IT-IT-T-C-A-T-C-A-G-C-C-T-T-C-T-IT-IC-IT |
| 51 | IA-IG-IT-IT-T-mC-A-T-mC-A-G-mC-mC-T-T-mC-T-IT-mIC-IT |
| 52 | IAs-IGs-ITs-ITs-Ts-Cs-As-Ts-Cs-As-Gs-Cs-Cs-Ts-Ts-Cs-Ts-ITs-ICs-IT |
| 53 | IAs-IGs-ITs-ITs-Ts-mCs-As-Ts-mCs-As-Gs-mCs-mCs-Ts-Ts-mCs-Ts-ITs-mICs-IT |
| 54 | IA-IG-IT-T-T-C-A-T-C-A-G-C-C-T-T-C-IT-IT-IC-IT |
| 55 | IA-IG-IT-T-T-mC-A-T-mC-A-G-mC-mC-T-T-mC-IT-IT-mIC-IT |
| 56 | IAs-IGs-ITs-Ts-Ts-Cs-As-Ts-Cs-As-Gs-Cs-Cs-Ts-Ts-Cs-ITs-ITs-ICs-IT |
| 57 | IAs-IGs-ITs-Ts-Ts-mCs-As-Ts-mCs-As-Gs-mCs-mCs-Ts-Ts-mCs-ITs-ITs-mICs-IT |
| 58 | ^{moe}A-^{moe}G-^{moe}T-^{moe}T-^{moe}T-^{moe}C-^{moe}A-^{moe}T-^{moe}C-^{moe}A-^{moe}G-^{moe}C-^{moe}C-^{moe}T-^{moe}T-^{moe}C-^{moe}T-^{moe}T- |
| | ^{moe}C-^{moe}T |
| 59 | ^{moe}A-^{moe}G-^{moe}T-^{moe}T-^{moe}T-^{moe}mC-^{moe}A-^{moe}T-^{moe}mC-^{moe}A-^{moe}G-^{moe}mC-^{moe}mC-^{moe}T-^{moe}T-^{moe}mC- |
| | ^{moe}T-^{moe}T-^{moe}mC-^{moe}T |
| 60 | ^{moe}As-^{moe}Gs-^{moe}Ts-^{moe}Ts-^{moe}Ts-^{moe}Cs-^{moe}As-^{moe}Ts-^{moe}Cs-^{moe}As-^{moe}Gs-^{moe}Cs-^{moe}Cs-^{moe}Ts-^{moe}Ts- |
| | ^{moe}Cs-^{moe}Ts-^{moe}Ts-^{moe}Cs-^{moe}T |
| 61 | ^{moe}As-^{moe}Gs-^{moe}Ts-^{moe}Ts-^{moe}Ts-^{moe}mICs-^{moe}As-^{moe}Ts-^{moe}mCs-^{moe}As-^{moe}Gs-^{moe}mCs-^{moe}mCs-^{moe}Ts- |
| | ^{moe}Ts-^{moe}mCs-^{moe}Ts-^{moe}Ts-^{moe}mCs-^{moe}T |
| 62 | ^{moe}As-^{moe}Gs-^{moe}Ts-^{moe}Ts-^{moe}Ts-^{moe}C-^{moe}A-^{moe}T-^{moe}C-^{moe}A-^{moe}G-^{moe}c-^{moe}C-^{moe}T-^{moe}Ts-^{moe}Cs-^{moe}Ts- |
| | ^{moe}Ts-^{moe}Cs-^{moe}T |
| 63 | ^{moe}As-^{moe}Gs-^{moe}Ts-^{moe}Ts-^{moe}Ts-^{moe}mC-^{moe}A-^{moe}T-^{moe}mC-^{moe}A-^{moe}G-^{moe}mC-^{moe}mC-^{moe}T-^{moe}Ts- |
| | ^{moe}mCs-^{moe}Ts-^{moe}Ts-^{moe}mCs-^{moe}T |
| 64 | ^{moe}As-^{moe}Gs-^{moe}Ts-^{moe}Ts-^{moe}Ts-^{moe}C-^{moe}A-^{moe}T-^{moe}C-^{moe}A-^{moe}G-^{moe}C-^{moe}C-^{moe}T-^{moe}T-^{moe}Cs-^{moe}Ts- |
| 65 | ^{moe}As-^{moe}Gs-^{moe}Ts-^{moe}Ts-^{moe}Ts-^{moe}mC-^{moe}A-^{moe}T-^{moe}mC-^{moe}A-^{moe}G-^{moe}mC-^{moe}mC-^{moe}T-^{moe}T- |
| | ^{moe}mCs-^{moe}Ts-^{moe}Ts-^{moe}mCs-^{moe}T |
| 66 | ^{moe}As-^{moe}Gs-^{moe}Ts-^{moe}Ts-^{moe}T-^{moe}C-^{moe}A-^{moe}T-^{moe}c-^{moe}A-^{moe}G-^{moe}C-^{moe}c-^{moe}T-^{moe}Ts-^{moe}Cs-^{moe}Ts- |
| | ^{moe}Ts-^{moe}Cs-^{moe}T |
| 67 | ^{moe}As-^{moe}Gs-^{moe}Ts-^{moe}Ts-^{moe}T-^{moe}mC-^{moe}A-^{moe}T-^{moe}mC-^{moe}A-^{moe}G-^{moe}mC-^{moe}mC-^{moe}T-^{moe}Ts- |
| | ^{moe}mCs-^{moe}Ts-^{moe}Ts-^{moe}mCs-^{moe}T |
| 68 | ^{moe}A-^{moe}G-^{moe}T-^{moe}T-^{moe}T-C-A-T-C-A-G-C-C-T-T-^{moe}C-^{moe}T-^{moe}T-^{moe}C-^{moe}t |
| 69 | ^{moe}A-^{moe}G-^{moe}T-^{moe}T-^{moe}T-mC-A-T-mC-A-G-mC-mC-T-T-^{moe}mC-^{moe}T-^{moe}T-^{moe}mC-^{moe}T |
| 70 | ^{moe}As-^{moe}Gs-^{moe}Ts-^{moe}Ts-^{moe}Ts-Cs-As-Ts-Cs-As-Gs-Cs-Cs-Ts-Ts-^{moe}Cs-^{moe}Ts-^{moe}Ts-^{moe}Cs-^{moe}T |
| 71 | ^{moe}As-^{moe}Gs-^{moe}Ts-^{moe}Ts-^{moe}Ts-mCs-AsTs-mCs-As-Gs-mCs-mCs-Ts-Ts-^{moe}mCs-^{moe}Ts-^{moe}Ts- |
| | ^{moe}mCs-^{moe}T |
| 72 | ^{moe}A-^{moe}G-^{moe}T-^{moe}T-^{moe}T-C-A-T-C-A-G-C-C-T-T-C-^{moe}T-^{moe}T-^{moe}C-^{moe}T |
| 73 | ^{moe}A-^{moe}G-^{moe}T-^{moe}T-^{moe}T-mC-A-T-mC-A-G-mC-mC-T-T-mC-^{moe}T-^{moe}t-^{moe}mC-^{moe}t |
| 74 | ^{moe}As-^{moe}Gs-^{moe}Ts-^{moe}Ts-^{moe}Ts-Cs-As-Ts-Cs-As-Gs-Cs-Cs-Ts-Ts-Cs-^{moe}Ts-^{moe}Ts-^{moe}Cs-^{moe}t |
| 75 | ^{moe}As-^{moe}Gs-^{moe}Ts-^{moe}Ts-^{moe}Ts-mCs-As-Ts-mCs-As-Gs-mCs-mCs-Ts-Ts-mCs-^{moe}Ts-^{moe}Ts- |
| | ^{moe}mCs-^{moe}T |
| 76 | ^{moe}A-^{moe}G-^{moe}T-^{moe}T-T-C-A-T-C-A-G-C-C-T-T-^{moe}C-^{moe}T-^{moe}T-^{moe}C-^{moe}T |
| 77 | ^{moe}A-^{moe}G-^{moe}T-^{moe}T-T-mC-A-T-mC-A-G-mC-mC-T-T-^{moe}C-^{moe}T-^{moe}t-^{moe}mC-^{moe}T |
| 78 | ^{moe}As-^{moe}Gs-^{moe}Ts-^{moe}Ts-Ts-Cs-As-Ts-Cs-As-Gs-Cs-Cs-Ts-Ts-^{moe}Cs-^{moe}Ts-^{moe}Ts-^{moe}Cs-^{moe}T |
| 79 | ^{moe}As-^{moe}Gs-^{moe}Ts-^{moe}Ts-Ts-mCs-As-Ts-mCs-As-Gs-mCs-mCs-Ts-Ts-^{moe}mCs-^{moe}Ts-^{moe}Ts- |
| | ^{moe}mCs-^{moe}T |
| 80 | ^{ome}A-^{ome}G-^{ome}T-^{ome}T-^{ome}T-^{ome}C-^{ome}A-^{ome}T-^{ome}C-^{ome}A-^{ome}G-^{ome}C-^{ome}C-^{ome}T-^{ome}T-^{ome}C-^{ome}T-^{ome}T- |
| | ^{ome}C-^{ome}T |
| 81 | ^{ome}A-^{ome}G-^{ome}T-^{ome}T-^{ome}T-^{ome}mC-^{ome}A-^{ome}T-^{ome}mC-^{ome}A-^{ome}G-^{ome}mC-^{ome}mC-^{ome}T-^{ome}T-^{ome}mC- |
| | ^{ome}T-^{ome}T-^{ome}mC-^{ome}T |
| 82 | ^{ome}As-^{ome}Gs-^{ome}Ts-^{ome}Ts-^{ome}Ts-^{ome}Cs-^{ome}As-^{ome}Ts-^{ome}Cs-^{ome}As-^{ome}Gs-^{ome}Cs-^{ome}Cs-^{ome}Ts-^{ome}Ts- |
| | ^{ome}Cs-^{ome}Ts-^{ome}Ts-^{ome}Cs-^{ome}T |
| 83 | ^{ome}As-^{ome}Gs-^{ome}Ts-^{ome}Ts-^{ome}Ts-^{ome}mCs-^{ome}As-^{ome}Ts-^{ome}mCs-^{ome}As-^{ome}Gs-^{ome}mCs-^{ome}mCs-^{ome}Ts- |
| | ^{ome}Ts-^{ome}mCs-^{ome}Ts-^{ome}Ts-^{ome}mCs-^{ome}T |
| 84 | ^{ome}As-^{ome}Gs-^{ome}Ts-^{ome}Ts-^{ome}Ts-^{ome}C-^{ome}A-^{ome}T-^{ome}C^{ome}A-^{ome}G-^{ome}C-^{ome}-C^{ome}-T^{ome}-Ts-^{ome}Cs-^{ome}Ts- |
| | ^{ome}Ts-^{ome}Cs-^{ome}T |
| 85 | ^{ome}As-^{ome}Gs-^{ome}Ts-^{ome}Ts-^{ome}Ts-^{ome}mC-^{ome}A-^{ome}T-^{ome}mC-^{ome}A-^{ome}G-^{ome}mC-^{ome}mC-^{ome}T-^{ome}Ts- |
| | ^{ome}mCs-^{ome}Ts-^{ome}Ts-^{ome}mCs-^{ome}T |
| 86 | ^{ome}As-^{ome}Gs-^{ome}Ts-^{ome}Ts-^{ome}Ts-^{ome}C-^{ome}A-^{ome}T-^{ome}C-^{ome}A-^{ome}G-^{ome}-C^{ome}-C^{ome}-T-^{ome}T-^{ome}Cs-^{ome}Ts- |
| | ^{ome}Ts-^{ome}Cs-^{ome}T |
| 87 | ^{ome}As-^{ome}Gs-^{ome}Ts-^{ome}Ts-^{ome}Ts-^{ome}mC-^{ome}A-^{ome}T-^{ome}mC-^{ome}A-^{ome}G-^{ome}mC-^{ome}mC-^{ome}T-^{ome}T- |
| | ^{ome}mCs-^{ome}Ts-^{ome}Ts-^{ome}-mCs-^{ome}T |
| 88 | ^{ome}As-^{ome}Gs-^{ome}Ts-^{ome}Ts-^{ome}T-^{ome}C-^{ome}A-^{ome}T-^{ome}C-^{ome}A-^{ome}G-^{ome}C-^{ome}C-^{ome}T-^{ome}Ts-^{ome}Cs-^{ome}Ts- |
| | Ts-^{ome}Cs-^{ome}T |
| 89 | ^{ome}As-^{ome}Gs-^{ome}Ts-^{ome}Ts-^{ome}T-^{ome}mC-^{ome}A-^{ome}T-^{ome}mC-^{ome}A-^{ome}G-^{ome}mC-^{ome}mC-^{ome}T-^{ome}Ts- |
| | ^{ome}mCs-^{ome}Ts-^{ome}Ts-^{ome}mCs-^{ome}T |
| 90 | ^{ome}A-^{ome}G-^{ome}T-^{ome}T-^{ome}T-C-A-T-C-A-G-C-C-T-T-^{ome}C-^{ome}T-^{ome}T-^{ome}C-^{ome}T |
| 91 | ^{ome}A-^{ome}G-^{ome}T-^{ome}T-^{ome}T-mC-A-T-mC-A-G-mC-mC-T-T-^{ome}mC-^{ome}T-^{ome}T-^{ome}mC-^{ome}T |
| 92 | ^{ome}As-^{ome}Gs-^{ome}Ts-^{ome}Ts-^{ome}Ts-Cs-As-Ts-Cs-As-Gs-Cs-Cs-Ts-Ts-^{ome}Cs-^{ome}Ts-^{ome}Ts-^{ome}Cs-^{ome}T |
| 93 | ^{ome}As-^{ome}Gs-^{ome}Ts-^{ome}Ts-^{ome}Ts-mCs-As-Ts-mCs-As-Gs-mCs-mCs-Ts-Ts-^{ome}mCs-^{ome}Ts-^{ome}Ts- |
| | mCs-^{ome}T |
| 94 | ^{ome}A-^{ome}G-^{ome}T-^{ome}T-^{ome}T-C-A-T-C-A-G-C-C-T-T-C-^{ome}T-^{ome}T-^{ome}C-^{ome}T |
| 95 | ^{ome}A-^{ome}G-^{ome}T-^{ome}T-^{ome}T-mC-A-T-mC-A-G-mC-mC-T-T-mC-^{ome}T-^{ome}T-^{ome}mC-^{ome}T |
| 96 | ^{ome}As-^{ome}Gs-^{ome}Ts-^{ome}Ts-^{ome}Ts-Cs-As-Ts-Cs-As-Gs-Cs-Cs-Ts-Ts-Cs-^{ome}Ts-^{ome}Ts-^{ome}Cs-^{ome}T |
| 97 | ^{ome}As-^{ome}Gs-^{ome}Ts-^{ome}Ts-^{ome}Ts-mCs-As-Ts-mCs-As-Gs-mCs-mCs-Ts-Ts-mCs-^{ome}Ts-^{ome}Ts- |
| | ^{ome}mCs-^{ome}T |
| 98 | ^{ome}A-^{ome}G-^{ome}T-^{ome}T-T-C-A-T-C-A-G-C-C-T-T-^{ome}C-^{ome}T-^{ome}T-^{ome}C-^{ome}T |
| 99 | A-^{ome}G-^{ome}T-^{ome}T-T-mC-A-T-mC-A-G-mC-mC-T-T-^{ome}mC-^{ome}T-^{ome}T-^{ome}mC-^{ome}T |
| 100 | ^{ome}As-^{ome}Gs-^{ome}Ts-^{ome}Ts-Ts-Cs-As-Ts-Cs-As-Gs-Cs-Cs-Ts-Ts-^{ome}Cs-^{ome}Ts-^{ome}Ts-^{ome}Cs-^{ome}T |
| 101 | ^{ome}As-^{ome}Gs-^{ome}Ts-^{ome}Ts-Ts-mCs-As-Ts-mCs-As-Gs-mCs-mCs-Ts-Ts-^{ome}mCs-^{ome}Ts-^{ome}Ts- |
| | ^{ome}mCs-^{ome}T |
| 105 | ^{ome}As-^{ome}Gs-Ts-Ts-Ts-^{ome}Cs-^{ome}As-Ts-^{ome}Cs-^{ome}As-^{ome}Gs-^{ome}C-^{ome}Cs-Ts-Ts-^{ome}mCs-Ts-Ts- |
| | ^{ome}mCs-T |
| 102 | A-G-T-T-T-C-A-T-C-A-G-C-C-T-T-C-T-T-C-T |
| 103 | A-G-T-T-T-C-A-T-C-A-G-C-C-T-T-C-T-T-C-T |

With IX: locked nucleic acid; ^{moe}χ: 2′-O-MOE modification; ^{ome}χ: 2′-O-Me modification; phosphorothioate linkage; italicised: PMO; italicized and underlined: PNA

In a preferred aspect, the ASO according to the invention is capable of inhibiting the replication of SARS-CoV-2 in the nucleus and in the cytoplasm. More particularly, the ASO is capable of decreasing the replication of SARS-CoV-2 by 10%, 20%, 30%, 40%, 50%, 60%, 70%, 80%, 90%, 91%, 92%, 93%, 94%, 95, 96%, 97%, 98%, 99% or 100% in comparison of its replication in absence of the ASO as measured by any method available to the person skilled in the art. More specifically, the replication can be assessed by one method as disclosed in the examples (cf. results from example 1).

In one particular aspect, the ASO according to the invention is capable of inhibiting the replication of one or more coronavirus, especially coronaviruses selected in the group of betacoronavirus. In a particular aspect, it is able to inhibit the replication of SARS-CoV-1, SARS-CoV-2, MERS-CoV, HCoV-OC43 and HCoV-HKU1. In another very particular aspect, it is able to inhibit the replication of SARS-CoV-1, SARS-CoV-2, and MERS-CoV. In a very particular aspect, it is able to inhibit the replication of SARS-CoV-2.

In a preferred aspect, the ASO of the invention comprises at least three distinct structural regions, namely a 5′ flank region (F), a gap region (G) and a 3′ flank region (F′). Besides, the F and F′ regions are composed of modified ribonucleotides (RNA*) whereas the G region is composed of deoxyribonucleotides, i.e. DNA or DNA-like nucleotides.

The ASOs are commonly used to inhibit a target nucleic acid via RNase H1 mediated degradation. Thus, the G region that comprises DNA or DNA-like nucleotides is responsible for RNAse H1 recruitment that leads to subsequent target nucleic acid cleavage. In contrast, the F and F′ regions comprise ribonucleotide sequence that are complementary to a target nucleic acid, i.e. two distinct regions of their target, and are thus responsible for the binding specificity to this target.

In particular aspects, the ASOs according to the invention consists or comprises a nucleotide sequence that corresponds to the following classical gapmer formula:

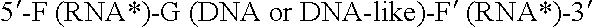

In a preferred aspect, the ASO of the invention consists of or comprises a molecule of formula 5′-F-G-F′-3′, where F region and F′ region independently comprise or consist of 1 to 10 ribonucleotides, preferably 2 to 9, 3 to 8, 4 to 7, 5 or 6 ribonucleotides, and G region comprises or consists of 6 to 20 deoxyribonucleotides, preferably 6 to 18, 6 to 14, 6 to 12, 6 to 10, 6 to 8, 10 to 16, 10 to 14, 12 to 16 or 14 deoxyribonucleotides. Optionally, F region and F′ region independently comprise or consist of 1, 2, 3, 4, 5, 6, 7, 8, 9 or 10 ribonucleotides, preferably 2, 3, 4, 5 or 6 ribonucleotides, more preferably 3, 4 or 5 ribonucleotides; and G region comprises or consists of 6, 7, 8, 9, 10, 11, 12, 13, 14, 15, 16, 17, 18, 19 or 20 deoxyribonucleotides.

The F and F′ regions usually comprise modified ribonucleotides that enhance the ASO binding affinity to its target nucleic acid and thus ensure a better selectivity for the knockdown strategy. In particular aspects, the ASO according to the invention comprises 2′-O-Me, 2′-O-MOE, 2′-F, 5-M, LNA, tcDNA, cET, PMO and/or PNA modified nucleotides in the in the F and F′ regions, preferably 2′-O-Me, 2′-O-MOE, 2′-F, 5-M, and/or LNA modified nucleotides, more preferably 2′-O-Me, 5-M, and/or LNA modified nucleotides. In a very specific aspect, F and F′ regions comprise 2′-O-Me or 2′-O-MOE modified nucleotides and some 5-M (i.e., the 5-methyl cytosines and uracils). Optionally, these modifications are combined with a fully phosphorothioate links.

In another very specific aspect, F and F′ regions comprise LNA modified nucleotides. Optionally, these modifications are combined with a fully phosphorothioate links.

In particular embodiments, the ASO according to the invention can be a headmer, a tailmer, a mixed wing ASO, an alternative flank ASO, a gap-breaker ASO (also called gap-disrupted gapmer) or comprises additional (D and D′) regions.

The terms “headmers” and “tailmers” refers to ASO capable of recruiting RNase H1 where one of the flank regions is missing, i.e., where only one of the ends of the oligonucleotide comprises affinity enhancing modified ribonucleotides. For headmers, the 3′ flank is missing (i.e., the 5′ flank comprises affinity enhancing modified nucleosides) and for tailmers, the 5′ flank is missing (i.e., the 3′ flank comprises affinity enhancing modified nucleosides). In particular embodiments, the ASO according to the invention consists or comprises a nucleotide sequence that corresponds to the following headmer (i) or (ii) tailmer formulas:

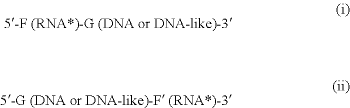

The terms “mixed wing ASO” refers to a LNA ASO wherein one or both of region F and F′ comprise a 2′ substituted nucleotide, such as a 2′ substituted nucleotide independently selected from the group consisting of 2′-O-alkyl-RNA units, 2′-O-methyl-RNA units, 2′-amino-DNA units, 2′-fluoro-DNA units, 2′-alkoxy-RNA, 2′-O-MOE units, 2′-O-Me units, arabino nucleic acid (ANA) units and 2′-fluoro-ANA units, such as 2′-O-MOE nucleotides.

In some aspects, wherein at least one of region F and F′, or both region F and F′ comprise at least one LNA nucleotide, the remaining nucleotides of region F and F′ are independently selected from the group consisting of 2′-O-MOE, 2′-O-Me and LNA. In some aspects, wherein at least one of region F and F′, or both region F and F′ comprise at least two LNA nucleotides, the remaining nucleotides of region F and F′ are independently selected from the group consisting of 2′-O-MOE, 2′-O-Me and LNA. In some mixed wing embodiments, one or both of region F and F′ may further comprise one or more deoxynucleotides. Some mixed wing ASO designs are disclosed in WO2008/049085 and WO2012/109395, the disclosure of which being incorporated herein by reference.

In some ASO, flank regions F and F′ may comprise both LNA and deoxynucleotides.

The terms “alternative flank ASO” refers to an ASO that comprises an alternating motif of LNA-DNA-LNA nucleotides. Alternative flank ASOs are thus LNA ASOs where at least one of the flanks (F or F′) comprises deoxynucleotides in addition to the LNA nucleotide(s). In some embodiments, at least one of region F or F′, or both region F and F′, comprise both LNA nucleotides and deoxynucleotides. In such embodiments, the flanking region. For F′, or both F and F′ comprise at least three nucleotides, wherein the 5′ and 3′ most nucleotides of the F and/or F′ region are LNA nucleotides. Besides, an alternating flank region may comprise up to 3 deoxynucleotides, such as 1 to 2 or 1 or 2 or 3 deoxynucleotides.

The terms “gap breaker ASO” or “gap-disrupted ASO” refers to an ASO wherein the G region comprise at least one 3′ endo modified nucleotides. There are numerous reports of the insertion of a modified nucleoside which confers a 3′ endo conformation into the gap region of ASOs, whilst retaining some RNase H1 recruitment capacity, see for example WO2013/022984, the disclosure of which being incorporated herein by reference.

Importantly, gap-breaker ASOs retain sufficient region of deoxynucleotides within the gap region to allow for RNase H1 recruitment. The ability of gap-breaker ASOs to recruit RNase H1 is typically sequence or even compound specific: see Rukov et al. 2015 Nucl. Acids Res. Vol. 43 pp. 8476-8487, which discloses gap-breaker ASOs recruiting RNase H1, which in some instances provide a more specific cleavage of the target RNA.

In addition, modified nucleotides used within the gap region of gap-breaker oligonucleotides may for example be modified nucleosides which confer a 3′endo conformation, such as 2′-O-Me or 2′-O-MOE nucleotides, or even beta-D LNA nucleotides (the bridge between 2′ and 4′ of the ribose sugar ring of a nucleotide is in beta conformation), such as beta-D-oxy LNA or ScET nucleosides.

Some gap region of gap-breaker or gap-disrupted ASOs have a deoxynucleotide at the 5′ end of the gap (adjacent to the 3′ ribonucleotide of region F), and a deoxynucleotide at the 3′ end of the gap (adjacent to the 5′ ribonucleotide of region F′). ASOs which comprise a disrupted gap typically retain a region of at least 3 or 4 deoxynucleotides at either the 5′ end or 3′ end of the gap region.

In some aspects, region G of a gap disrupted ASO comprises at least 6 deoxynucleotides, such as 6, 7, 8, 9, 10, 11, 12, 13, 14, 15, 16, 17, 18, 19, 20 or 21 deoxynucleotides. Also, the deoxynucleotides may be or may optionally be interspersed with one or more modified nucleotides, with the proviso that the gap region G is capable of mediating an effective RNase H1 recruitment.

The ASO according to the invention may in some embodiments comprise or consist of the nucleotide sequence of the classical formula, i.e. F-G-F′, and further comprising 5′ and/or 3′ nucleotides. The further 5′ and/or 3′ nucleotides may or may not be fully complementary to the target nucleic acid. Such further 5′ and/or 3′ nucleotides may be referred to as region D′ and D″ herein.

The addition of region D′ or D″ may be used for the purpose of joining the nucleotide sequence of the ASO to a conjugate moiety or another functional group. When used for joining the ASO sequence with a conjugate moiety, one peripheral region, i.e. D′ and/or D″, can serve as a biocleavable linker (described below). Alternatively, it may be used to provide exonuclease protection or for ease of synthesis or manufacture.

Region D′ and D″ can be attached to the 5′ end of region F (i), the 3′ end of region F′ (ii) or both (iii), respectively to generate designs of the following formulas:

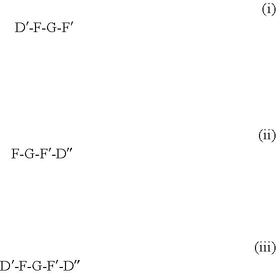

In this instance, the F-G-F′ is the ASO portion of the oligonucleotide and region D′ or D″ constitute a separate part of the oligonucleotide. Region D′ or D″ may independently comprise or consist of 1, 2, 3, 4 or 5 additional nucleotides, which may be complementary or non-complementary to the target nucleic acid. The nucleotide adjacent to the F or F′ region is not a sugar-modified nucleotide, such as a DNA or RNA or base modified versions of these.

As described above, the D′ or D′ region may serve as a nuclease susceptible biocleavable linker.

In some aspects, the additional 5′ and/or 3′ end nucleotides are linked with phosphodiester linkages. Nucleotide based biocleavable linkers suitable for use as region D′ or D″ are notably disclosed in WO2014/076195, which include by way of example a phosphodiester linked DNA dinucleotide. The use of biocleavable linkers in poly-oligonucleotide constructs is disclosed in WO2015/113922, where they are used to link multiple antisense constructs within a single oligonucleotide.

In a particular aspect, the present invention relates to any of the ASO or combination of different ASOs described above. The present invention relates to any of the ASO described above, a pharmaceutical composition comprising it and their use as a drug.

### Pharmaceutical Composition and its Uses

The present invention relates to a pharmaceutical composition comprising any of the ASOs described above, and its uses as a drug, especially for the treatment and prevention of SARS-CoV-2 infection. More preferably, the pharmaceutical composition comprises the ASO comprising, essentially consisting in or consisting in a sequence set forth in SEQ ID NO: 1, in particular any ASO with modification(s), optionally any ASO as disclosed in Table A (SEQ ID NOs: 24 and 45-103) or a combination thereof.

In a preferred aspect, the additional therapeutic agent is one or several other ASOs targeting SARS-CoV-2 genome. Optionally, the other ASOs may have a target site in 5′ UTR, ORF1a, ORF1b, S, ORF3a, E, M, ORF6, ORF7a, ORF7b, ORF8, N, ORF10, or 3′ UTR regions, more preferably in 5′UTR, ORF1ab-nsp1, ORF1ab-nsp12, ORF1ab-FSE, or N. Optionally, the other ASOs can be any ASO already available in the art, for instance any of the ASOs disclosed in Hedge et al, 2021, supra; Quemener and Galibert, 2021, supra; Zhu et al., 2022, supra; Vora et al., 2022, supra; Li et al, 2021, supra; Zhang et al, 2021, supra; Lulla et al, 2021, supra; Dauksaite et al, 2022, supra; Dhorne-Pollet et al, 2022, supra; WO2022/031410; WO2021/207641 and WO2021/195025, the disclosure of which being incorporated herein by reference. Alternatively, the pharmaceutical composition may further comprise one or several ASOs comprising, essentially consisting in or consisting in any of the sequences of SEQ ID NO: 2-21, preferably any of the sequences of SEQ ID NO: 2-6, still more preferably a sequence of SEQ ID NO: 2. In a particular aspect, the pharmaceutical composition is any one as disclosed above.

Optionally, the pharmaceutical composition further comprises an additional therapeutic agent. The additional therapeutic agent can be non-exhaustively an antiviral drug, an anti-inflammatory agent or a coronavirus treatment. In a particular aspect, the additional therapeutic agent can be selected in the group consisting of, but not limited to, paxlovid, remdesivir, favipiravir, molnupiravir, dexamethasone, bamlanivimab, casirivimab, imdevimab, convalescent plasma, PF-07321332, anakinra, regdanvimab, tocilizumab, nirmatrelvir, bebtelovimab, tixagevimab, cilgavimab, interferons and any combination thereof. Examples of suitable antiviral agents include, but are not limited to, baloxavir, marboxil, oseltamivir, anamivir, vidarabine, acyclovir, ganciclovir, zidovudine, didanosine, zalcitabine, lamivudine, saquinavir, ritonavir, indinavir, nelfinavir, ribavirin, amantadine, rimantadine, paxlovid, remdesivir, favipiravir, and molnupiravir.

The pharmaceutical compositions contemplated herein may include a pharmaceutically acceptable carrier in addition to the active ingredient(s).

The term “pharmaceutically acceptable carrier” is meant to encompass any carrier (e.g., support, substance, solvent, etc.) which does not interfere with effectiveness of the biological activity of the active ingredient(s) and that is not toxic to the host to which it is administered. For example, for parenteral administration, the active compounds(s) may be formulated in a unit dosage form for injection in vehicles such as saline, dextrose solution, serum albumin and Ringer's solution.

The pharmaceutical composition can be formulated as solutions in pharmaceutically compatible solvents or as emulsions, suspensions or dispersions in suitable pharmaceutical solvents or vehicle, or as pills, tablets or capsules that contain solid vehicles in a way known in the art. Formulations of the present invention suitable for oral administration may be in the form of discrete units as capsules, sachets, tablets or lozenges, each containing a predetermined amount of the active ingredient; in the form of a powder or granules; in the form of a solution or a suspension in an aqueous liquid or non-aqueous liquid; or in the form of an oil-in-water emulsion or a water-in-oil emulsion. Formulations suitable for parenteral administration conveniently comprise a sterile oily or aqueous preparation of the active ingredient which is preferably isotonic with the blood of the recipient. Every such formulation can also contain other pharmaceutically compatible and nontoxic auxiliary agents, such as, e.g. stabilizers, antioxidants, binders, dyes, emulsifiers or flavoring substances. The formulations of the present invention comprise an active ingredient in association with a pharmaceutically acceptable carrier therefore and optionally other therapeutic ingredients. The carrier must be “acceptable” in the sense of being compatible with the other ingredients of the formulations and not deleterious to the recipient thereof. The pharmaceutical compositions are advantageously applied by injection or intravenous infusion of suitable sterile solutions or as oral dosage by the digestive tract or as inhalation. Methods for the safe and effective administration of most of these agents are known to those skilled in the art. In addition, their administration is described in the standard literature.

The compositions can be presented in unit dosage form and may be prepared by any methods well known in the art of pharmacy. The amount of the one or more ASOs which can be combined with a carrier material to produce a single dosage form will vary depending upon the host being treated, the particular mode of administration, and/or the type and severity of coronavirus infection. The amount of active ingredient which can be combined with a carrier material to produce a single dosage form will generally be that amount of the ASO which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

Actual dosage levels of the active ingredients (e.g., ASO molecules) in the pharmaceutical compositions of this disclosure may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular ASO of the present disclosure employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the ASO of the disclosure employed in the pharmaceutical composition at levels lower than that required in order to achieve the desired therapeutic effect and gradually increase the dosage until the desired effect is achieved.

In general, a suitable daily dose of the ASO of the disclosure is the amount of the compound that is the lowest dose effective to produce a therapeutic effect. Such an effective dose generally depends upon the factors described above.

Preferably, the ASO or the pharmaceutical composition comprising it as disclosed herein are administered at about 10 μg/kg to about 20 mg/kg, more preferably at about 1 mg/kg to about 15 mg/kg, even more preferably at about 5 mg/kg to about 10 mg/kg.

In a particular aspect, the ASO or the pharmaceutical composition comprising it as disclosed herein can be administered once, twice or more. For example, the effective dose of the active compound (e.g., ASO molecule) may be administered at appropriate intervals throughout the day, or over the course of days, weeks, months. For instance, the dose may be administered 1 to 3 times a day, or once a day, once a week, once a month or once every 2 month. For instance, five administrations can be carried out. A prime administration may be followed by a boost administration. For instance, the administration can be spaced by 3 weeks or 2 weeks and could be adapted to reach a defined virus load and/or to the medical conditions of the subject to be treated.

The administration route for the ASO and the pharmaceutical composition comprising it as disclosed herein may be oral, respiratory, parenteral, systemic, intravenous, subcutaneous, topical, rectal, transdermal, intradermal, nasal, intramuscular, intraperitoneal, and the like. Preferably, the administration route is parenterally, e.g., intravenously, subcutaneously, intradermally, or intramuscularly. More preferably, the administration route is the respiratory route, with nasal or mouth inhalation and nebulization, which may be used for a deeper diffusion into the lungs.

The disclosed ASO can be administered alone or in combination with one or more additional different ASO, coronavirus treatment agents and/or antiviral agents. The additional coronavirus treatment agents and/or antiviral may be a small molecule (e.g., a nucleoside analog or a protease inhibitor) or a biologic (e.g., an antibody or peptide). Examples of suitable coronavirus treatment agents include, but are not limited to, paxlovid, remdesivir, favipiravir, molnupiravir, dexamethasone, bamlanivimab, casirivimab, imdevimab, convalescent plasma, PF-07321332, anakinra, regdanvimab, tocilizumab, nirmatrelvir, bebtelovimab, tixagevimab, cilgavimab, interferons and any combination thereof. Examples of suitable antiviral agents include, but are not limited to, baloxavir, marboxil, oseltamivir, anamivir, vidarabine, acyclovir, ganciclovir, zidovudine, didanosine, zalcitabine, lamivudine, saquinavir, ritonavir, indinavir, nelfinavir, ribavirin, amantadine, rimantadine, paxlovid, remdesivir, favipiravir, and molnupiravir. Accordingly, the present invention further relates to
a pharmaceutical composition comprising an ASO as disclosed above, preferably the ASO comprising, essentially consisting in or consisting in a sequence set forth in SEQ ID NO: 1, optionally an additional therapeutic agent as disclosed above, and a pharmaceutically acceptable carrier for use as a drug;
a pharmaceutical composition comprising an ASO as disclosed above, preferably the ASO comprising, essentially consisting in or consisting in a sequence set forth in SEQ ID NO: 1, in combination with an additional therapeutic agent as disclosed above, and a pharmaceutically acceptable carrier for use as a drug;
a pharmaceutical composition comprising an ASO as disclosed above, preferably the ASO comprising, essentially consisting in or consisting in a sequence set forth in SEQ ID NO: 1, optionally an additional therapeutic agent as disclosed above, and a pharmaceutically acceptable carrier for use in the treatment or prevention of a viral infection by a coronavirus, preferably an infection by a severe acute respiratory syndrome-related coronavirus (SARS-CoV), more preferably a SARS-CoV-2 or MERS-CoV virus infection;
a pharmaceutical composition comprising an ASO as disclosed above, preferably the ASO comprising, essentially consisting in or consisting in a sequence set forth in SEQ ID NO: 1, and a pharmaceutically acceptable carrier in combination with an additional therapeutic agent as disclosed above for use in the treatment or prevention of a viral infection by a coronavirus, preferably an infection by a severe acute respiratory syndrome-related coronavirus (SARS-CoV), more preferably a SARS-CoV-2 or MERS-CoV virus infection;
a product or kit containing an ASO as disclosed above, preferably the ASO comprising, essentially consisting in or consisting in a sequence set forth in SEQ ID NO: 1, and an additional therapeutic agent as disclosed above as a combined preparation for simultaneous, separate or sequential use, in particular in the treatment or prevention of a viral infection by a coronavirus, preferably an infection by a severe acute respiratory syndrome-related coronavirus (SARS-CoV), more preferably a SARS-CoV-2 or MERS-CoV virus infection;
a combined preparation which comprises an ASO as disclosed above, preferably the ASO comprising, essentially consisting in or consisting in a sequence set forth in SEQ ID NO: 1, and an additional therapeutic agent as disclosed above for simultaneous, separate or sequential use, in particular in the treatment or prevention of a viral infection by a coronavirus, preferably an infection by a severe acute respiratory syndrome-related coronavirus (SARS-CoV), more preferably a SARS-CoV-2 or MERS-CoV virus infection;
the use of an ASO as disclosed above, preferably the ASO comprising, essentially consisting in or consisting in a sequence set forth in SEQ ID NO: 1, for the manufacture of a medicament for the treatment or prevention of a viral infection by a coronavirus in a patient in need thereof, preferably an infection by a severe acute respiratory syndrome-related coronavirus (SARS-CoV), more preferably a SARS-CoV-2 or MERS-CoV virus infection;
the use of an ASO as disclosed above, preferably the ASO comprising, essentially consisting in or consisting in a sequence set forth in SEQ ID NO: 1, and an additional therapeutic agent as disclosed above, for the manufacture of a medicament for the treatment or prevention of a viral infection by a coronavirus in a patient in need thereof, preferably an infection by a severe acute respiratory syndrome-related coronavirus (SARS-CoV), more preferably a SARS-CoV-2 or MERS-CoV virus infection;
a method for treating or preventing a viral infection by a coronavirus in a patient in need thereof, preferably an infection by a severe acute respiratory syndrome-related coronavirus (SARS-CoV), more preferably a SARS-CoV-2 or MERS-CoV virus infection, comprising administering an effective amount of an ASO as disclosed above, preferably the ASO comprising, essentially consisting in or consisting in a sequence set forth in SEQ ID NO: 1;
a method for treating or preventing a viral infection by a coronavirus in a patient in need thereof, preferably an infection by a severe acute respiratory syndrome-related coronavirus (SARS-CoV), more preferably a SARS-CoV-2 or MERS-CoV virus infection, comprising administering an effective amount of an ASO as disclosed above, preferably the ASO comprising, essentially consisting in or consisting in a sequence set forth in SEQ ID NO: 1, and administering an effective amount of an additional therapeutic agent as disclosed above; and
a method for treating or preventing a viral infection by a coronavirus in a patient in need thereof, preferably an infection by a severe acute respiratory syndrome-related coronavirus (SARS-CoV), more preferably a SARS-CoV-2 or MERS-CoV virus infection, comprising administering an effective amount of a pharmaceutical composition comprising an ASO as disclosed above, preferably the ASO comprising, essentially consisting in or consisting in a sequence set forth in SEQ ID NO: 1, and an additional therapeutic agent as disclosed above.

In particular aspect, the ASO comprising, essentially consisting in or consisting in a sequence set forth in SEQ ID NO: 1 is with modification(s), optionally is any ASO as disclosed in Table A (SEQ ID NOs: 24 and 45-103) or a combination thereof.

### Coronaviruses and Coronavirus Infections

According to the invention, the coronavirus is an alphacoronavirus or a betacoronavirus, preferably a betacoronavirus. More specifically, the pharmaceutical composition is suitable for preventing or treating an infection by several betacoronaviruses. Optionally, the pharmaceutical composition is suitable for preventing or treating an infection by an alphacoronavirus.

In one aspect, the pharmaceutical composition as disclosed herein is for use for preventing or treating an infection by an alphacoronavirus or a betacoronavirus. In a preferred aspect, the betacoronavirus is selected from the group consisting of SARS-CoV-1, SARS-CoV-2, MERS-CoV, HCoV-OC43 and HCoV-HKU1, more specifically the group consisting of SARS-CoV-2, and MERS-CoV. In a preferred embodiment, the pharmaceutical composition as disclosed herein is for preventing or treating an infection by SARS-CoV-1, SARS-CoV-2, MERS-CoV, HCoV-OC43 and HCoV-HKU1. Preferably, the pharmaceutical composition as disclosed herein is for preventing or treating an infection by SARS-CoV-2, and MERS-CoV. More preferably, the pharmaceutical composition as disclosed herein is for preventing or treating an infection by SARS-CoV-2.

According to the invention, the subject to be treated may be a mammal, and it includes humans and non-human mammals. In some embodiments, the subject is a human, such as an adult human. In a preferred embodiment, the subject according to the invention to be treated is a subject at risk of contracting a coronavirus infection or a subject infected by a coronavirus. More preferably, the subject according to the invention is a subject aged 65 years or older, a subject having a cancer or having had a cancer, a subject being obese, in particular with severe obesity (body mass index [BMI] of 40 or higher [CDC-HCSP BMI>30]), a subject being diabetic, a subject having a hypertension, a subject having a sarcoidosis, a subject being immunocompromised, a subject who lives in a nursing home or long-term care facility, a subject with chronic lung disease or moderate to severe asthma, lung fibrosis, a subject who has serious heart conditions, a subject with chronic kidney disease undergoing dialysis and/or a subject with liver diseases. Optionally, the subject can be a subject with a stable comorbidity factor, for instance, stable cancer patients, chronic obstructive pulmonary disease (COPD) patients, stable patients with comorbidity as obesity or renal dialysis.

Many conditions can cause a person to be immunocompromised (including cancer treatment, smoking, bone marrow or organ transplantation, immune deficiencies, poorly controlled HIV or AIDS, prolonged use of corticosteroids and other immune weakening medications).

### EXAMPLES

### Example A

### Results

### Example A-1

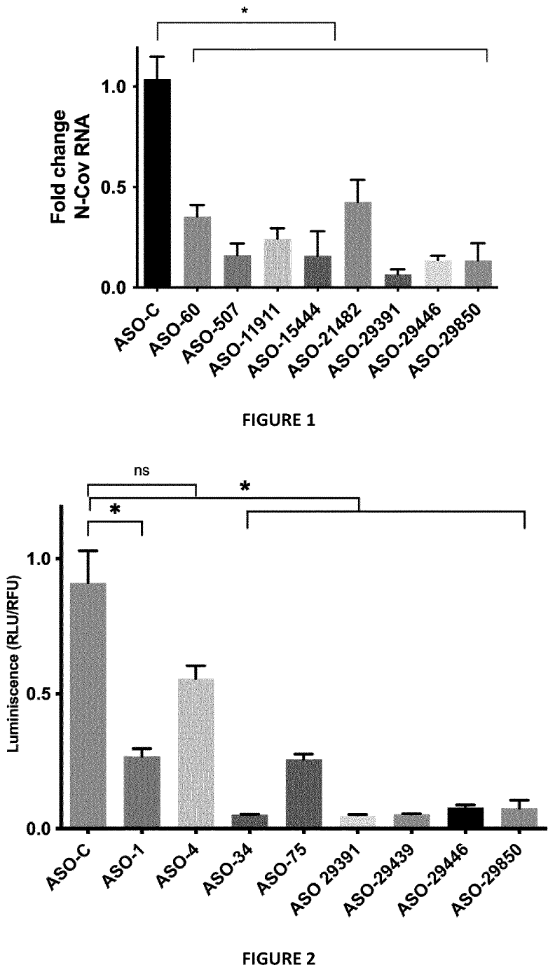

FIGS. 1 and 2 show the comparison of ASOs designed to target 5′UTR, ORF1ab gene, N gene or 3′ UTR of the SARS-CoV-2 genome to inhibit SARS-CoV-2 replication. The percentages of inhibition for each ASO are provided in Table 3.

The efficiency to inhibit the SARS-CoV-2 was firstly evaluated by RT-qPCR to measure N-Cov RNA quantity in HEK-293T/ACE2 cells in culture (FIG. 1). The 5 best ASOs (SEQ ID NO: 1-5, Table 1) decrease significatively the viral replication between 66% and 94% compared to Control ASO (ASO-C FC=1; p<0.05). The best ASO candidate tested was the ASO-29391 with a reduction of 94% in comparison with ASO-C.

In addition, the efficiency to inhibit the SARS-CoV-2 was also evaluated by measuring the luminescence by co-transfection of ASOs and the minigenome luciferase reporter including the 5′UTR, nucleocapsid gene and 3′UTR regions of SARS-CoV-2 genome (FIG. 2). The minigenome luciferase reporter is an original method developed by the inventors to screen ASO candidates (Plasmid construct). Again, one of the most efficient ASO to reduce the minigenome gene expression was ASO-29391 with a reduction of 95% in comparison with ASO-C. The ASO-1, ASO-4, ASO-75 targeting the 5′UTR region are in average less efficient than the ASO targeting the N gene or the 3′UTR (ASO-29391, ASO-29439 and ASO-29446 for the N gene and ASO-29850 for the 3′UTR).

One of the most efficient ASO to reduce the minigenome gene expression was the ASO-29391. The ASO-1, ASO-4, ASO-75 targeting the 5′UTR region are in average less efficient than the ASO targeting the 3′UTR (ASO-29391 and others ASO-29446 and ASO-29850). The best ASO candidate tested was the ASO-29391 with a reduction of 95% in comparison with ASO-C.

The analysis of viral genomic (positive strand) and antigenomic (negative strand) by qPCR of ORF1b region after ASO transfection of the infected cells revealed significant decrease of both viral gRNA strands (positive and negative), as already observed with other ASOs as described in Dhorne-Pollet et al, 2022, Supplement FIG. 9B.

### Example A-2

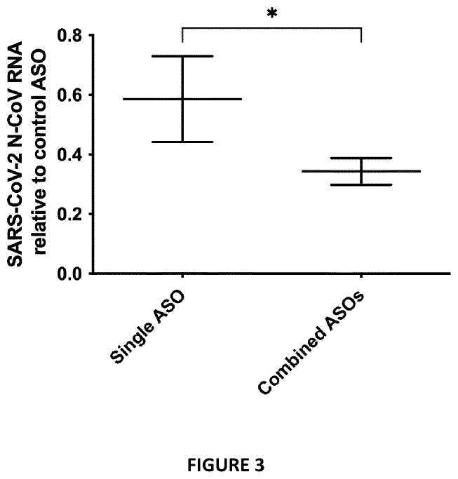

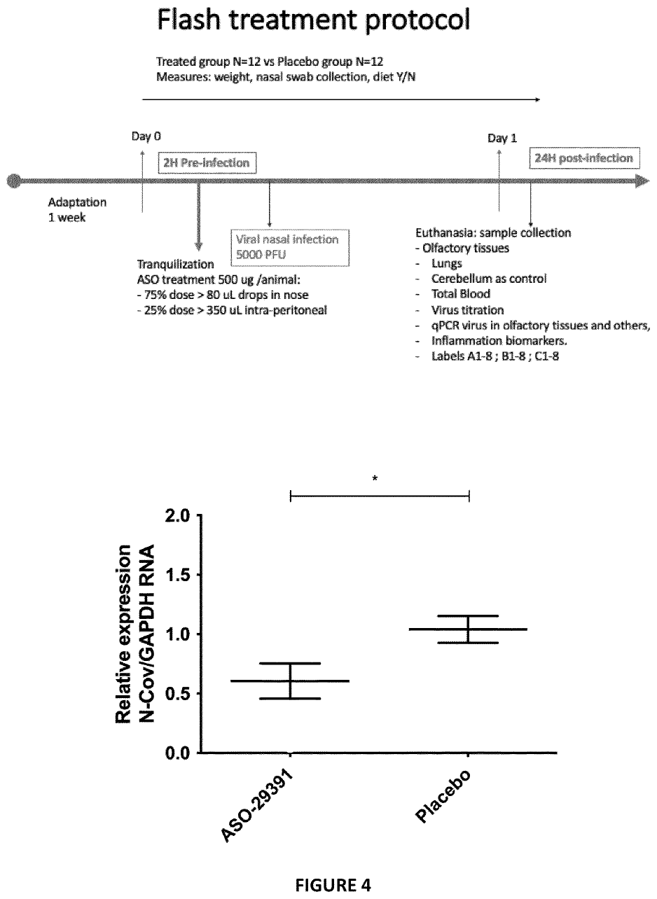

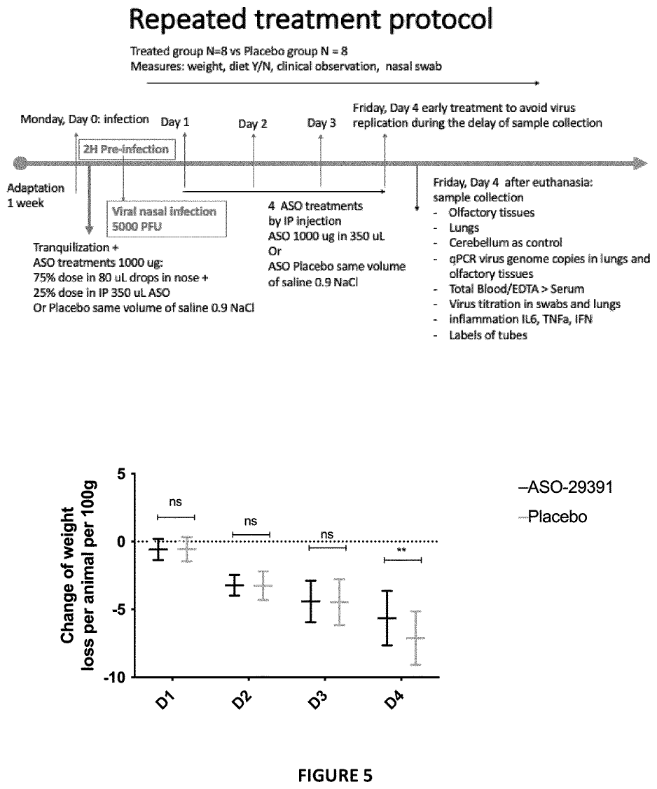

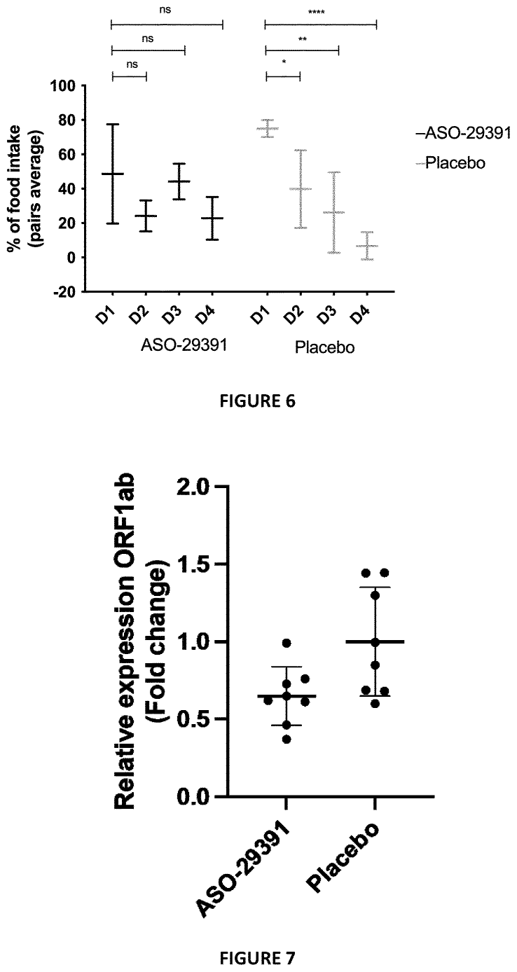

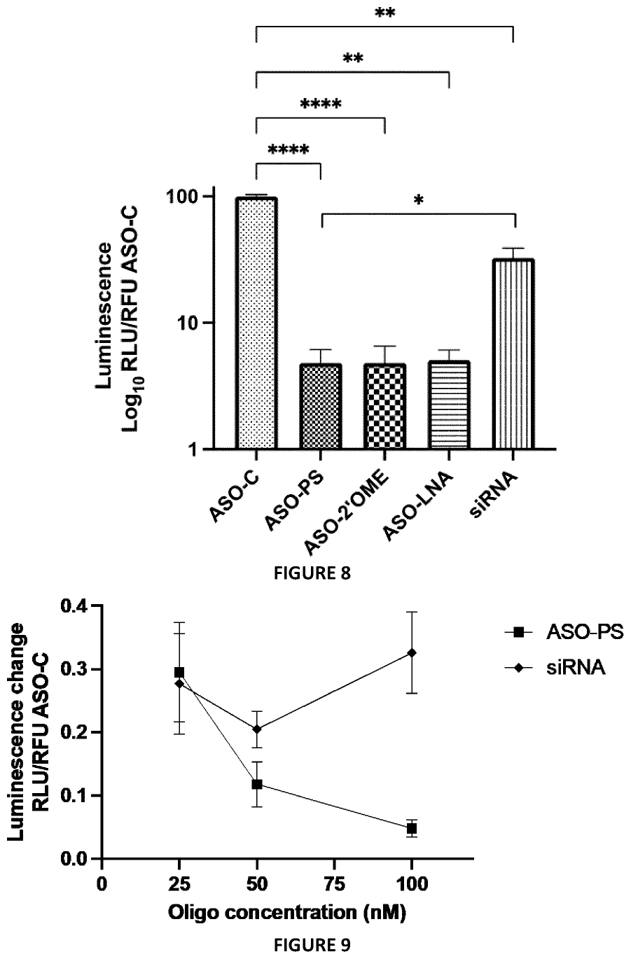

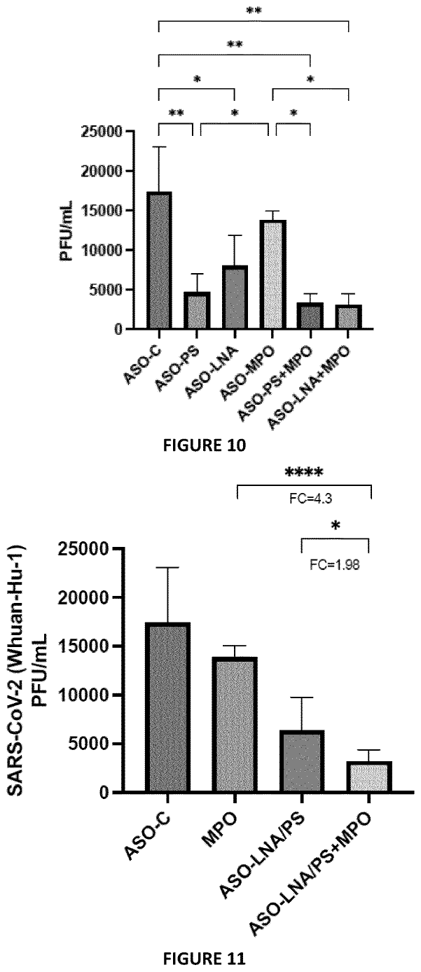

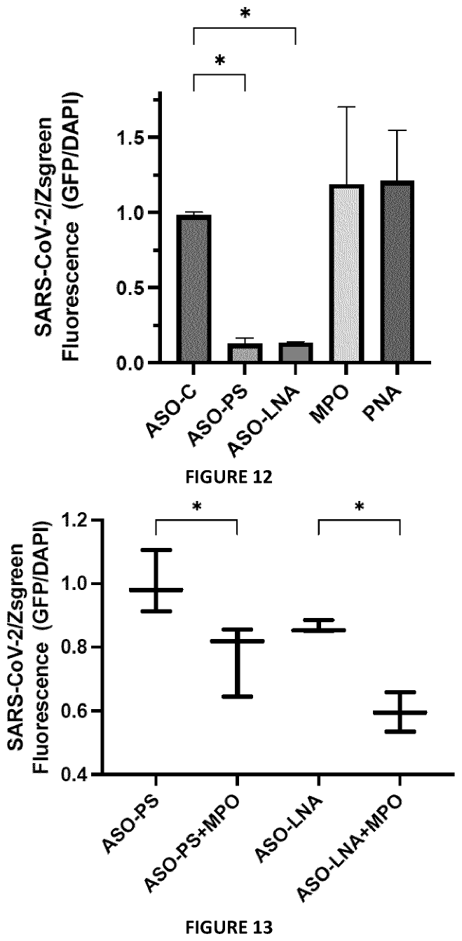

The inventors assessed the efficiency of a combination of two ASOs in comparison to the same single ASO. Results are shown in FIG. 3. In average, it was observed that some combinations of two ASOs showed a synergic effect and improve the efficiency of one or both ASOs included in the combination. For instance, ASO-29391 could be efficiently associated with an ASO targeting ORF1ab such as ASO-15444 or with an ASO targeting N gene or 3′UTR region such as ASO-29439, ASO-29446 or ASO-29850. In particular, the two best combinations were: ASO-29391+ASO-507 and ASO-29391+ASO-15444. The combination of two ASOs such as ASO-29391+ASO-507 leads to a SARS-CoV-2 replication (quantified by qPCR N-CoV transcript) which is in average 59% lower than with a single ASO.

### Example A-3

Next, the inventors assessed the capacity of ASO to prevent or decrease an infection by SARS-CoV-2 virus. Animals were treated with ASO 2 hours before infection and the effects were assessed 24 hours post-infection by measuring N-CoV RNA quantity by RT-qPCR. Results were shown in FIG. 4.

A clear decrease of viral genome copies in the treated group in comparison to the placebo group was observed (p<0.05). The treatment with ASO-29391 reduced the viral load by 42% in the olfactory tissues.

### Example A-4

The inventors further assessed the capacity of ASO to treat an infection by SARS-CoV-2 virus. In this context, after viral infection, animals were treated with 4 doses of ASO. Results were shown in FIGS. 5-8.

The treated group recovered with a significant weight increase at Day 4 in comparison to the placebo group (p<0.05) (FIG. 5). This result is confirmed by the food intake (FIG. 6). Indeed, food intake is maintained in the group treated with ASO whereas the group treated with placebo showed a diminution of food intake that increases from Day 1 to Day 4. More specifically, the food intake decreased more rapidly in the placebo group after infection (p<0.05−p<0.001) than in the treated group which showed variability but no significant change of food intake Day 3 and Day 4 vs Day 1 (p>0.05).

The inventors observed that the treatment with ASO-29391 significantly reduced the relative viral RNA as assessed by measuring ORF1ab copies by RT-PCR by 35% on Day 4 in the nasal mucosa (p<0.05) (FIG. 7).

Finally, in the lungs, less infected than nasal mucosa, the ASO treatment reduced the viral load by 10% on Day 4 in comparison with the Placebo group.

### Material and Methods

### ASO Design

The principle of the ASO activity to block and/or cleaved the RNA target is described (FIG. 1A, Dhorne-Pollet et al. 2022) and experimentally demonstrated in Dhorne-Pollet et al. 2022 and other previous studies targeting other SARS-CoV-2 regions than in the present patent (Lulla et al., 2021; Rosenke et al., 2021; Vora et al., 2022; Zhu et al., 2022). The design of the ASOs was based on the reference SARS-CoV-2 genome (ID #: NC_045512.2=GenBank MN908947; Wu et al. 2020) (FIG. 1B, Dhorne-Pollet et al. 2022). The initial strategy of the inventors was based on the selection of the regions of the viral genome involved early in the replication process: 5′UTR, ORF1a, ORF1b, gene N and 3′UTR. The 5′UTR of the viral genome is necessary to initiate replication, while ORF1a and ORF1b code for the replicase polyproteins and were thus very interesting targets in order to block replication of the virus at early stages (V'kovski et al., 2020). The N gene region is interesting because it is the most transcribed and expressed gene of the viral genome. The N-ORF10-3′UTR region is interesting to target in order to block the transcription and regulation process of the replication with stem loops and domains which seem to interact with the 5′UTR-ORF1ab region.

### Analysis of the Secondary Structure of the SARS-CoV-2 RNA Genome

In order to better evaluate the suitability of ASO candidates, the inventors performed secondary structure prediction of viral genome target segments with the MFold tool, which can analyze up to 4000 nt-long sequences. They computed the 5′UTR region (1-265), ORF1ab and N-3′UTR in sequences of approximately 2000 nt flanking the target sites of their ASO candidates. The binding sites for ASO candidates were then analyzed according to the presence of single and double-stranded regions.

### ASO Prediction

Using the viral target sequences previously described, the inventors computed complementary oligonucleotides by considering many well-known parameters such as GC %, Melting-temperature (Tm), to avoid track of nucleotides such as GGGG, and other parameters according to their expertise such as AT % and secondary target sequences on the viral genome.

### Sequence Conservation of ASO Targets in SARS-CoV-2 Variants of Concern (VOC)

Finally, the short list of ASO which were tested as efficient were tested in silico against the main identified VOC. The inventors performed i) an alignment against all the SARS-CoV-2 sequences available on the NCBI database, using the Betacoronavirus BLAST tool (over 13,000 SARS-CoV-2 genome sequences); ii) an alignment against the SARS-CoV-2 sequences of the main VOC identified (Wuhan, Alpha, Beta, Delta, Mu, Omicron BA.2, BA.4, BA.5, BA.2.75, BQ.1, BQ.1.1, XBB) available on the GISAID database, using the BLASTN tool (GISAID: tracking of variants hCoV-19). (See Table 2)

### ASO Synthesis and Preparation Before Use

In order to maximize the probability of efficiency of the selected antisense oligonucleotide candidates in knocking-down the viral RNA sequences, the inventors applied a validated method commonly used for designing ASOs used in RNA silencing. They designed 20-mer antisense oligonucleotides (ASOs) with 100% phosphorothioate (PS) linkages (Suppl. FIG. 1A, Dhorne-Pollet et al. 2022). This chemical modification is widely used in antisense oligonucleotide synthesis for stability and ultimately efficient cleavage of viral RNA targets by RNase H1 (FIG. 1A, Dhorne-Pollet et al. 2022). PS is a backbone modification in which one non-bridging oxygen atom in the α-phosphate group is replaced by sulfur (Suppl. FIG. 1A, Dhorne-Pollet et al. 2022), thereby conferring higher resistance to nucleases. The ASOs were further described in Table 1.

### Antiviral Activity ASO Screening by Minigenome Reporter Assay

In order to select efficient targets among 5′UTR, nucleocapsid N, ORF10 and 3′UTR genomic sequences of the viral genome, a minigenome reporter assay expressing the firefly luciferase reporter gene was used. Briefly, the plasmid construct (Luc-N cloned into pVITRO2-neo-GFP/LacZ, between Bgl2 and Nhe1 restriction sites (SEQ ID NO: 104) expressed complete 5′UTR, firefly Luciferase reporter gene, nucleocapsid N, ORF10 and 3′UTR genomic sequences. In short, 150.000 HEK293T/ACE2 cells were seeded in 24 well transparent cell culture plates (Corning). After 24 hours when cells reached 80 percent confluence, cells were transfected using Lipofectamine 2000 (Invitrogen) with a plasmid mixture containing 0.4 μg of pVITRO2-neo-GFP/LacZ/Luc-N minigenome, 0.04 μg of control plasmid pCMV-tdTomato expressing a red fluorescent protein dsRed as normalizer, and 200 nM of ASO control (ASO-C) or SARS-CoV-2 directed ASOs with 2 μl/well. Signal intensity was measured in white bottom 96 well plates (Corning) using TECAN Infinite® 200 PRO plate reader (Tecan Life Sciences, Männedorf, Switzerland). Luminescence was determined using Luciferase assay reagent (Promega) and DsRed Fluorescence was measured under 540 nm excitation and 580 nm emission.

### Antiviral Activity ASO Screening by Infective SARS-CoV-2 Virus in HEK-293T/ACE2 Cells

In order to validate ASOs activity with infective SARS-CoV-2 virus, HEK-293T/ACE2 cells were seeded into 12-well plates, at a density of 2×10⁵ cells/well. After reaching 80% confluence, cells were transfected with 100 nM ASO-C or ASOs directed to the SARS-CoV-2 genome using 4 μl/well Lipofectamine 2000 (Invitrogen). cells infected with SARS-CoV-2 Three hours post-transfection, were BetaCoV/France/IDF0372/2020 strain at MOI=0.1. 24 hours post-infection, cells were collected for RNA extraction. Cells were washed twice with PBS and total RNA was extracted using TRIzol LS Reagent (Life Technologies) according to the manufacturer's recommendations. RNA yield and purity was monitored with a NanoDrop ND-1000 spectrophotometer. Reverse transcription (RT) was performed with the SuperScript IV first-strand synthesis system (Invitrogen). One μg total RNA and 50 ng random hexamer primers (Life Technologies) were mixed, denatured for 5 min at 65° C., spun-down, and stored on ice before adding the reaction mix, according to manufacturer's instructions. RT was carried out at 23° C. for 10 min and then 50° C. for 45 min. The reaction was stopped by heating at 80° C. for 10 min. After cDNA synthesis, RNA was degraded by incubating with 4 U RNase H1 for 20 min at 37° C. The reaction was stopped by heating at 70° C. for 10 min and cDNAs were stored at −20° C. until use. To quantify SARS-CoV-2 RNA copies, we used primers for the N-CoV gene designed by the Centers for Disease Control and Prevention USA: forward (2019-nCOV_N1-F): 5′-GACCCCAAAATCAGCGAAAT (SEQ ID NO: 22); reverse (2019-nCOV_N1-R9): 5′-TCTGGTTACTGCCAGTTGAATCTG (SEQ ID NO: 23). This set of primers have been validated for sensitivity and efficiency (Vogels et al. 2020). N-CoV RT-qPCR analysis was performed with SybrGreen power PCR master mix reagent (Applied Biosystems) in StepOne as previously described (Dhorne-Pollet et al 2022). In short, mixtures were incubated at 95° C. for 10 min, followed by 40 cycles of 95° C. for 15 s and 60° C. for 1 min. The qPCR results of target gene N-CoV were analyzed by the 2-AACT method (Livak et al. 2001) and expressed as fold-change relative to ASO-C.

### ASO Selection for In Vivo Pre-Clinical

Finally, ASO-29391 was chosen in the short list of the best evaluated ASO according to the experimental screening procedure performed in vitro. The ASO-29391 was one of the most efficient according to different tests performed and in addition, its genomic viral target on the nucleocapsid gene (N-CoV) is well conserved among all the main VOCs observed in spring-autumn 2022 in Europe (Wuhan, Alpha, Beta, Delta, Mu, Omicron BA.2, BA.4, BA.5, BA.2.75) (See Table 2).

### Pre-Clinical Trial 1: One Flash Treatment

A flash treatment using ASO-29391 was evaluated in vivo on 12 treated Golden Hamsters compared to 12 Golden Hamsters receiving a placebo (Isotonic saline, 0.9% NaCl). The treatment was administrated 2 hours before the experimental infection. The total dose was 500 μg/animal including 75% of the dose by drops into the nose after tranquilization of the animal and 25% of the dose by intra-peritoneal injection. The ASO was resuspended into purified isotonic saline in 80 μL for nasal instillation and in 500 μL for intra-peritoneal injection. Then, 2 hours after ASO administration, drops including a viral load of 5000 PFU was administrated to the animals of the two groups treated and placebo.

Twenty four hours after the treatment, the animals were euthanatized and the nasal epithelium, lungs and other tissues were collected for subsequent analyses. In a preliminary analysis, we performed total RNA extraction on the nasal epithelium for quantifying N-CoV copies to evaluate the treatment effect.

The inventors' studies follow protocols approved by the ANSES/EnvA/UPEC Ethics Committee (CE2A-16) and authorized by the French ministry of research under the number APAFIS #25384-2020041515287655 in accordance with the French and European regulations.

### Pre-Clinical Trial 2: Repeated Treatments 1Time/Day×5 Days During SARS-CoV-2 Infection

A five-days repeated treatment using ASO-29391 was evaluated in vivo on 8 treated Golden Hamsters compared to 8 Golden Hamsters receiving a placebo (purified isotonic saline). On Day 0, the prophylactic dose is given before experimental infection, and during infection the treatment dose was repeated each day until day 4 post-infection.

Day 0 (DO): the treatment was administrated 2 hours before the experimental infection. The total dose was 1000 μg/animal including 75% of the dose by drops into the nose after tranquilization of the animal and 25% of the dose by intra-peritoneal injection. The ASO was resuspended into purified isotonic saline in 80 μL for nasal instillation and in 350 μL for intra-peritoneal injection. Then, 2 hours after ASO administration, drops including a viral load of 5000 PFU was administrated to the animals of the two groups treated and placebo.

Day 1-Day 4 post-infection: all the animals of the treated group received an intra-peritoneal injection either of 1000 μg of ASO-29391 in 350 μl of saline in the treated group or the same volume of saline in the placebo group.

Day 4: after the treatment in the morning, all the animals were euthanatized and the tissues collected as in pre-clinical trial 1.

In a preliminary analysis, the inventors performed a total RNA extraction as described above on the nasal mucosa collected at Day 4 for quantifying the ORF1ab viral RNA expression number of copies to analyze the treatment effect. The reference gene UEB2D2 was used to normalized all the ORF1ab expressions. The relative expression of ORF1ab was calculated by comparing the ASO treated group vs. the placebo group with a fold change adjusted to 1. The qPCR results were analyzed by the 2^{-ΔΔCT} method (Livak et al. 2001) and expressed as fold-change relative to ORF1ab expression in placebo group.

The inventors' studies follow protocols approved by the ANSES/EnvA/UPEC Ethics Committee (CE2A-16) and authorized by the French ministry of research under the number APAFIS #25384-2020041515287655 in accordance with the French and European regulations.

TABLE 1
ASO sequences

| | | | | | Target positions |
|---|---|---|---|---|---|
| SEQ ID NO | | | ASO sequences 5′ | SARS-CoV-2 | 5′-3′ (Ref genome |
| | (PS) | ASO ID | to 3′ | Target | NC_045512.2) |
| 1 | 24 | ASO-29391 | AGTTTCATCAGCCTTCTTCT | N | 29391; 29410 |
| 2 | 25 | ASO-15444 | CATATAGTGAACCGCCACAC | ORF1ab-nsp12 | 15444; 15463 |
| 3 | 26 | ASO-29446 | CAGCAGGAAGAAGAGTCACA | N | 29446; 29465 |
| 4 | 27 | ASO-507 | CTCAACCATAACATGACCAT | ORF1ab-nsp1 | 507; 526 |
| 5 | 28 | ASO-60 | TTAAAGTTCGTTTAGAGAACA | 5′UTR | 60; 80 |
| 6 | 29 | ASO-13503 | TACTAGTGCCTGTGCCGCAC | ORF1ab-FSE | 13503; 13522 |
| 7 | 30 | ASO-1 | GGAAGGTATAAACCTTTAAT | 5′UTR | 1; 20 |
| 8 | 31 | ASO-4 | CTGGGAAGGTATAAACCTTT | 5′UTR | 4; 23 |
| 9 | 32 | ASO-34 | CAAGAGATCGAAAGTTGGTT | 5′UTR | 34; 53 |
| 10 | 33 | ASO-75 | CAGCCACACAGATTTTAAAG | 5′UTR | 75; 94 |
| 11 | 34 | ASO-7127 | CCACTAAGACAAACACTACA | ORF1ab | 7127;7146 |
| 12 | 35 | ASO-11911 | CCCACAATTTAGATGATGAT | ORF1ab-nsp7 | 11911; 11930 |
| 13 | 36 | ASO-13241 | CAACGGCAGTACAGACAACA | ORF1ab-nsp10 | 13241; 13260 |
| 14 | 37 | ASO-13396 | CACAACTACAGCCATAACCT | ORF1ab-FSE | 13396; 13415 |
| 15 | 38 | ASO-18798 | TAGGTTACCTGTAAAACCCC | ORF1ab-nsp14 | 18779; 18798 |
| 16 | 39 | ASO-19722 | CTACATCAACACCATCAACT | ORF1ab | 19722; 19741 |
| 17 | 40 | ASO-20972 | GAGACAAAGTCATTAAGATC | ORF1ab-nsp16 | 20953; 20972 |
| 18 | 41 | ASO-21482 | AGAAGAGATAAAATCATATC | ORF1ab-nsp16 | 21463; 21482 |
| 19 | 42 | ASO-29439 | AAGAAGAGTCACAGTTTGCT | N | 29439; 29458 |
| 20 | 43 | ASO-29574 | CGTAAACGGAAAAGCGAAAA | ORF10 | 29574; 29593 |
| 21 | 44 | ASO-29850 | CATTCTCCTAAGAAGCTAT | 3′UTR | 29850; 29868 |

TABLE 2
2 ASOs with 100% homology with at least 9 out of the 10 human SARS-CoV-2
variants with a single mismatch for ASO-29391 on VOC Delta (no more active)
and two mismatches for ASO-15444 on VOC Delta and Omicron XBB

| SEQ | | | | | | | | BLAST- |
|---|---|---|---|---|---|---|---|---|
| ID | BLAST | BLAST- | BLAST- | BLAST- | BLAST- | BLAST- | BLAST- | OMICRON |
| NO: | Wuhan | ALFA | BETA | GAMMA | DELTA | LAMBDA | MU | BA.1 |
| 1 | 100% | 100% | 100% | 100% | 1 | 100% | 100% | 100% |
| | | | | | mismatch | | | |
| 2 | 100% | 100% | 100% | 100% | 1 | 100% | 100% | 100% |
| | | | | | mismatch | | | |

| SEQ | BLAST- | BLAST- | BLAST- | BLAST | BLAST |
|---|---|---|---|---|---|
| ID | OMICRON | OMICRON | OMICRON | OMICRON | OMICRON |
| NO: | BA.4 | BA.5 | BQ1 | BQ1.1 | XBB |
| 1 | 100% | 100% | 100% | 100% | 100% |
| 2 | 100% | 100% | 100% | 100% | 1 |
| | | | | | mismatch |

TABLE 3
ASOs and percentages of inhibition versus ASO-Control

| | | Percentage of minigenome | Percentage of |
|---|---|---|---|
| SEQ | | luciferase reporter | N-CoV qPCR |
| ID | | Luc-N reduction | reduction vs |
| NO: | ASO ID | vs ASO-Control (%) | ASO-Control (%) |
| 1 | ASO-29391 | 95 | 94 |
| 2 | ASO-15444 | N/A | 85 |
| 3 | ASO-29446 | 91 | 87 |
| 4 | ASO-507 | N/A | 84 |
| 5 | ASO-60 | 98 | 66 |
| 6 | ASO-13503 | N/A | 50 |
| 7 | ASO-1 | 71 | N/A |
| 8 | ASO-4 | 39 | N/A |
| 9 | ASO-34 | 94 | N/A |
| 10 | ASO-75 | 72 | N/A |
| 11 | ASO-7127 | N/A | 48 |
| 12 | ASO-11911 | N/A | 77 |
| 13 | ASO-13241 | N/A | 42 |
| 14 | ASO-13396 | N/A | 35 |
| 15 | ASO-18798 | N/A | 46 |
| 16 | ASO-19722 | N/A | 70 |
| 17 | ASO-20972 | N/A | 17 |
| 18 | ASO-21482 | N/A | 59 |
| 19 | ASO-29439 | 94 | N/A |
| 20 | ASO-29574 | 92 | N/A |
| 21 | ASO-29850 | 92 | 87 |

### Example B

### Results

### Example B-1: Comparison of the Efficiency of the ASO-29391 with Different Chemistries: PS, 2′OME, LNA and of an siRNA Having the Same Sequence

Test reporter vectors containing SARS-CoV-2 nucleocapsid coding region included in a minigenome N-Luc plasmid was used in this assay. The luminescent reporter system allows the inventors to directly quantify which chemistry of ASO is the more efficient to downregulate the viral RNA target.

### Methods

Cells were seeded at 2 10⁵ per well (in 24 well plates) and 24 h after, minigenome N-Luc plasmid at 400 ug per well, tomato Dsred plasmid for transfection control at 40 ug per well, selected ASO at 100 nM, transfected with 2 μL lipofectamine 2000 per well. 24 h after transfection fluorescence was read in 100 uL of trypsinized cells. After, 100 uL of the Luciferase detection system Promega was added and luciferase was measured in a TECAN plate reader. In 96 well COSTAR white bottom plates.

### Results

The results are shown in FIG. 8. ASO-29391 with chemistries PS, 2′OME, LNA were significantly more efficient to downregulate targeted RNA than ASO-Control and siRNA having the same sequence. Even if siRNA is active against the target; its effect is significantly less efficient than RNAse H1 dependent ASO-29391 (ASO-PS, ASO-2′OME, ASO-LNA).

In addition, the efficiency of ASO-29391 PS and of the siRNA having the same sequence has been compared at increasing doses. The results are shown in FIG. 9. At 50 nM and 100 nM, the efficiency of ASO-29391 PS is higher than siRNA, suggesting saturation of siRNA-mediated gene silencing pathway at 25 nM.

### Example B-2: Effect of Different ASO Combinations with Two Different Mechanisms of Action

The aim is to test the efficiency of a mixture 50/50 of RNaseH-dependent (ASO-PS/ASO-LNA) and steric-blocking ASO (MPO). The hypothesis is that the addition of steric blocking ASO such as MPO will increase the efficiency by avoiding the risk of saturation by the ASO of RNASe H1 dependent mechanism of action.

### Methods

Viral titration assay (TCID): VeroE6/TMPRSS2 cells (3×10⁵) cells were transfected with 100 nM oligonucleotides with lipofectamin 2000 (3 ul/well) and infected with SARS-CoV-2 (Whuan Hu-1) at MOI=0.1 in 12 well plates (Corning). When a mixture of ASO was used, 50 nM of the first ASO and 50 nM of the second ASO were used so as to maintain a whole concentration of 100 nM oligonucleotides. Viral titers were determined by the TCID₅₀ method performed in 96 well plates. In VeroE6 cells (1×10⁴ cells per well) by 3 days incubation. The cytopathic effect was determined by crystal violet staining. Finaly, raw TCID₅₀ results were transformed in PFU/ml.

SARS-CoV-2/Zsgreen virus assay: SARS-CoV-2/Zsgreen virus at MOI 0.1, incubated with described oligonucleotides for 48 hours. When a single ASO is used, a concentration of 100 nM was used. When a mixture of ASO was used, 50 nM of the first ASO and 50 nM of the second ASO were used so as to maintain a whole concentration of 100 nM oligonucleotides. Fluorescence was measured in plate reader TECAN for GFP at 480/9 nm excitation and 530/20 nm emission in 96 well black flat bottom plates.

### Results

The results of Viral titration assay (TCID) are shown in FIGS. 10 and 11. ASO-29391 PS is significantly better than ASO-29391MPO for SARS-CoV-2 antiviral activity in vitro by transfection infection challenge in Vero cells. Moreover, the combination of ASO-29391 PS and ASO-29391 MPO or ASO-29391 LNA and ASO-29391 MPO is better than ASO-29391 MPO alone or than ASO-29391 PS or LNA alone. When comparing the results of the effect of the combination with the ASO alone, it is important to remember that half of the concentration of each ASO was used for the combination.

In FIG. 11, the data obtained with ASO PS and LNA were pooled. It can be observed that ASO chemistries PS and LNA act synergically with steric blocking ASO MPO or PNA. When comparing the results of the effect of the combination with the ASO alone, it is important to remember that the concentration was constant and each ASO was used in a ratio 50:50 for the combination.

Mixtures of ASO directed to the same RNA target region but with different mechanisms of action increase the antiviral activity. The effect of the mixture was tested by a second method, namely SARS-CoV-2/Zsgreen virus assay. The RNase H1 dependent ASO PS and LNA were compared alone against the steric-blocking ASO MPO and PNA (FIG. 12). Boosted transfection induce a strong decrease in fluorescence in SARS-CoV-2/Zsgreen infected VeroE6/TMPRSS2 cells with both RNase H1 dependent ASOs (PS and LNA) while Steric block oligonucleotides (MPO and PNA) fail to induce any decrease. Conversely, ASO mixture is 4.3 and 1.98 times better than ASO MPO, PNA or PS-LNA alone as show in FIG. 13. For each combination ASO-PS+MPO and ASO-LNA+MPO, there is a significant improvement in the antiviral activity as confirmed by fluorescent virus measurements.

To conclude, ASO-PS and ASO-LNA are significantly more efficient than ASO MPO for SARS-CoV-2 antiviral activity in vitro by transfection infection challenge in Vero cells. The mixture of RNase H1 dependent ASO and steric-blocking ASO MPO or PNA is significantly more efficient than each ASO alone at the same concentration (i.e., 50 nM/50 nM vs 100 nM ASO alone). In average, ASO mixture is 4.3 and 1.98 times better than ASO MPO or ASO PS-LNA alone, respectively, to decrease viral titer. Similar results were obtained by the fluorescent virus.

ASOs with chemistries PS and LNA act synergically with ASO MPO or PNA. Consequently, a mixture of ASOs directed to the same RNA target region with different mechanisms of action increases the antiviral activity.

### Material of Example B

ASO-29391 was tested with different chemistries, namely PS (SEQ ID NO: 24), 2′OME (SEQ ID NO: 105), LNA (SEQ ID NO: 48), PNA (SEQ ID NO: 103) and MPO (SEQ ID NO: 102). The molecules were described in Table 4.

TABLE 4

| SEQ ID | |
|---|---|
| NO | ASO-29391 |
| 24 | As-Gs-Ts-Ts-Ts-Cs-As-Ts-Cs-As-Gs-Cs- |
| | Cs-Ts-Ts-Cs-Ts-Ts-Cs-Ts |
| 48 | IAs-IGs-ITs-ITs-Ts-Cs-As-Ts-Cs-As-Gs- |
| | Cs-Cs-Ts-Ts-Cs-ITs-ITs-ICs-IT |
| 105 | ^{ome}As-^{ome}Gs-Ts-Ts-Ts-^{ome}Cs-^{ome}As-Ts-^{ome}Cs- |
| | ^{ome}As-^{ome}Gs-^{ome}C-^{ome}Cs-Ts-Ts-^{ome}mCs-Ts-Ts- |
| | ^{ome}mCs-T |
| 102 | A-G-T-T-T-C-A-T-C-A-G-C-C-T-T-C-T-T-C-T |
| 103 | A-G-T-T-T-C-A-T-C-A-G-C-C-T-T-C-T-T-C-T |

With IX: locked nucleic acid;
^{ome}X: 2′-O-Me modification;
s: phosphorothioate linkage;
italicised: PMO;
italicized and underlined: PNA

The siRNA having the same sequence than ASO-29391 has the following sequence:

(SEQ ID NO: 106)
sense strand: 5′-GAAGAAGGCTGATGAAACUTTT-3′;
(SEQ ID NO: 107)
antisense strand:
5′-AGUUUCAUCAGCCUUCUUCdTdT-3′.

### REFERENCES

Barrey, E., Burzio, V., Dhorne-pollet, S., and Delmas, B. (2020). Think different with RNA therapy: Can antisense oligonucleotides be used to inhibit replication and transcription of SARS-Cov-2? Preprints. doi: 10.20944/preprints202004.0412. v1
Dhorne-Pollet, S., Fitzpatrick, C., Da Costa, B., Bourgon, C., Eléouët, J., Meunier, N., . . . . Barrey, E. (2022). Antisense oligonucleotides targeting ORF1b block replication of severe acute respiratory syndrome coronavirus 2 (SARS-CoV-2). Frontiers in Microbiology, 13 (October), 1-13. https://doi.org/10.3389/fmicb.2022.915202
Livak, K. J., and Schmittgen, T. D. (2001). Analysis of relative gene expression data using real-time quantitative PCR and the 2-δδCT method. Methods 25, 402-408.
Lulla, V., Wandel, M. P., Bandyra, K. J., Ulferts, R., Wu, M., Dendooven, T., et al. (2021). Targeting the conserved stem loop 2 motif in the SARS-CoV-2 genome. J. Virol. 95: e0066321. doi: 10.1128/JVI.00663-21
Rosenke, K., Leventhal, S., Moulton, H. M., Hatlevig, S., Hawman, D., Feldmann, H., et al. (2021). Inhibition of SARS-CoV-2 in Vero cell cultures by peptide-conjugated morpholino oligomers. J. Antimicrob. Chemother. 76, 413-417. doi: 10.1093/jac/dkaa460
V'kovski, P., Kratzel, A., Steiner, S., Stalder, H., and Thiel, V. (2020). Coronavirus biology and replication: Implications for SARS-CoV-2. Nat. Rev. Microbiol. 19, 155-170. doi: 10.1038/s41579-020-00468-6
Vogels, C. B. F., Brito, A. F., Wyllie, A. L., Fauver, J. R., Ott, I. M., Kalinich, C. C., et al. (2020). Analytical sensitivity and efficiency comparisons of SARS-CoV-2 RT-qPCR primer-probe sets. Nat. Microbiol. 5, 1299-1305. doi: 10.1038/s41564-020-0761-6
Wu, F., Zhao, S., Yu, B., Chen, Y.-M., Wang, W., Song, Z.-G., et al. (2020). A new coronavirus associated with human respiratory disease in China. Nature 579, 265-269. doi: 10.1038/s41586-020-2008-3
Zhu, C., Lee, J. Y., Woo, J. Z., Xu, L., Nguyenla, X., Yamashiro, L. H., et al. (2022). An intranasal ASO therapeutic targeting SARS-CoV-2. Nat. Commun. 13:4503. doi: 10.1038/s41467-022-32216-0

1-21. (canceled)

22. An antisense oligonucleotide (ASO), wherein the ASO comprises a sequence set forth in SEQ ID NO: 1 and further comprises one or several chemical modifications, said modifications being selected from modifications of nucleotide and/or modifications of internucleotide linkage.

23. The ASO of claim 22, wherein the ASO acts via RNase H1 mediated degradation or wherein the ASO acts via blocking the target sequence.

24. The ASO of claim 22, wherein the internucleotide linkage modification is: a phosphorothioate (PS) linkage modification or PS linkage modifications for at least for 3 to 5 terminal 5′ and/or 3′ nucleotides.

25. The ASO of claim 22, wherein the ASO comprises nucleotide modifications selected from locked nucleic acid (LNA) modification, 2′ O-methoxyethyl(2′-O-MOE) modification, 2′ O-methyl(2′-O-Me) modification, 2′-fluoro (2′-F) modification, phosphorodiamidate morpholino oligomer (PMO) modification, peptide-conjugated phosphoroamidate morpholino oligomer (PPMO) modification, peptide nucleic acid modification (PNA), constrained ethyl (cEt) bridged nucleic acid (BNA) modification, tricyclo-DNA (tcDNA) modification and 5-methyl (5-M) modification or combinations thereof.

26. The ASO of claim 25, wherein the ASO comprises nucleotide modifications selected from the group consisting of LNA modifications, 2′-O-MOE modifications and 2′-O-Me modifications, for at least 3 to 5 terminal 5′ and/or 3′ nucleotides; and/or the ASO comprises 5-methyl cytosine and/or 5-methyl uracil modifications.

27. The ASO of claim 22, wherein the ASO is selected from an ASO comprising SEQ ID NO: 1, 24, 45-103, or 105.

28. The ASO of claim 22, wherein all nucleotides are phosphorodiamidate morpholino oligomer (PMO) or peptide nucleic acid oligomer (PNA).

29. A pharmaceutical composition, wherein the pharmaceutical composition comprises the ASO of claim 22 and a pharmaceutically acceptable carrier.

30. The pharmaceutical composition of claim 29, wherein the composition further comprises one or more ASO complementary to SARS-CoV-2 genome or an ASO having a target site in 5′ UTR, ORF1a, ORF1b, S, ORF3a, E, M, ORF6, ORF7a, ORF7b, ORF8, N, ORF10, or 3′ UTR regions of the SARS-CoV2 genome.

31. The pharmaceutical composition of claim 30, wherein the composition further comprises one or more ASO comprising one of SEQ ID NOs: 2 to 21.

32. The pharmaceutical composition of claim 31, wherein the composition further comprises one ASO comprising a sequence set forth in SEQ ID NO: 2, SEQ ID NO: 3, SEQ ID NO: 4, or SEQ ID NO: 19.

33. The pharmaceutical composition of claim 29, wherein the pharmaceutical composition comprising two or more ASOs with different chemistries that exhibit complementary and synergic effect.

34. The pharmaceutical composition of claim 33, wherein the pharmaceutical composition comprises one ASO comprising phosphorothioate (PS) linkage modification and/or nucleotide modifications selected from the group consisting of LNA modifications and 2′-O-Me modifications and one ASO in which all nucleotides are phosphorodiamidate morpholino oligomer (PMO) or peptide nucleic acid oligomer (PNA).

35. The pharmaceutical composition of claim 33, wherein the ASOs comprise a sequence set forth in SEQ ID NO: 1; or wherein the pharmaceutical composition comprises one ASO selected from the group consisting of SEQ ID NOs: 24, 45-101 and 105 and one ASO selected from the group consisting of SEQ ID NOs: 102 and 103.

36. A method of treating a viral infection by a coronavirus comprising the administration of an ASO according to claim 22 or a pharmaceutical composition comprising said ASO and a pharmaceutically acceptable carrier to a subject in need of treatment.

37. The method of claim 36, wherein said pharmaceutical composition is administered by injection, inhalation, or nebulization.

38. The method of claim 36, wherein said viral infection is caused by a severe acute respiratory syndrome-related coronavirus (SARS-CoV), SARS-CoV-2 or MERS-CoV.


SEQUENCE LISTING (107 sequences)

SEQ ID NO: 1 - DNA, 20 bp, synthetic construct
  source 1..20: mol_type = other DNA
agtttcatcagccttcttct

SEQ ID NO: 2 - DNA, 20 bp, synthetic construct
  source 1..20: mol_type = other DNA
catatagtgaaccgccacac

SEQ ID NO: 3 - DNA, 20 bp, synthetic construct
  source 1..20: mol_type = other DNA
cagcaggaagaagagtcaca

SEQ ID NO: 4 - DNA, 20 bp, synthetic construct
  source 1..20: mol_type = other DNA
ctcaaccataacatgaccat

SEQ ID NO: 5 - DNA, 21 bp, synthetic construct
  source 1..21: mol_type = other DNA
ttaaagttcgtttagagaaca

SEQ ID NO: 6 - DNA, 20 bp, synthetic construct
  source 1..20: mol_type = other DNA
tactagtgcctgtgccgcac

SEQ ID NO: 7 - DNA, 20 bp, synthetic construct
  source 1..20: mol_type = other DNA
ggaaggtataaacctttaat

SEQ ID NO: 8 - DNA, 20 bp, synthetic construct
  source 1..20: mol_type = other DNA
ctgggaaggtataaaccttt

SEQ ID NO: 9 - DNA, 20 bp, synthetic construct
  source 1..20: mol_type = other DNA
caagagatcgaaagttggtt

SEQ ID NO: 10 - DNA, 20 bp, synthetic construct
  source 1..20: mol_type = other DNA
cagccacacagattttaaag

SEQ ID NO: 11 - DNA, 20 bp, synthetic construct
  source 1..20: mol_type = other DNA
ccactaagacaaacactaca

SEQ ID NO: 12 - DNA, 20 bp, synthetic construct
  source 1..20: mol_type = other DNA
cccacaatttagatgatgat

SEQ ID NO: 13 - DNA, 20 bp, synthetic construct
  source 1..20: mol_type = other DNA
caacggcagtacagacaaca

SEQ ID NO: 14 - DNA, 20 bp, synthetic construct
  source 1..20: mol_type = other DNA
cacaactacagccataacct

SEQ ID NO: 15 - DNA, 20 bp, synthetic construct
  source 1..20: mol_type = other DNA
taggttacctgtaaaacccc

SEQ ID NO: 16 - DNA, 20 bp, synthetic construct
  source 1..20: mol_type = other DNA
ctacatcaacaccatcaact

SEQ ID NO: 17 - DNA, 20 bp, synthetic construct
  source 1..20: mol_type = other DNA
gagacaaagtcattaagatc

SEQ ID NO: 18 - DNA, 20 bp, synthetic construct
  source 1..20: mol_type = other DNA
agaagagataaaatcatatc

SEQ ID NO: 19 - DNA, 20 bp, synthetic construct
  source 1..20: mol_type = other DNA
aagaagagtcacagtttgct

SEQ ID NO: 20 - DNA, 20 bp, synthetic construct
  source 1..20: mol_type = other DNA
cgtaaacggaaaagcgaaaa

SEQ ID NO: 21 - DNA, 19 bp, synthetic construct
  source 1..19: mol_type = other DNA
cattctcctaagaagctat

SEQ ID NO: 22 - DNA, 20 bp, synthetic construct
  source 1..20: mol_type = other DNA
gaccccaaaatcagcgaaat

SEQ ID NO: 23 - DNA, 24 bp, synthetic construct
  source 1..24: mol_type = other DNA
tctggttactgccagttgaatctg

SEQ ID NO: 24 - DNA, 20 bp, synthetic construct
  source 1..20: mol_type = other DNA
  modified_base 1..20: mod_base = OTHER; note = phosphorothioate internucleotide linkages
agtttcatcagccttcttct

SEQ ID NO: 25 - DNA, 20 bp, synthetic construct
  source 1..20: mol_type = other DNA
  modified_base 1..20: mod_base = OTHER; note = phosphorothioate internucleotide linkage
catatagtgaaccgccacac

SEQ ID NO: 26 - DNA, 20 bp, synthetic construct
  source 1..20: mol_type = other DNA
  modified_base 1..20: mod_base = OTHER; note = phosphorothioate internucleotide linkage
cagcaggaagaagagtcaca

SEQ ID NO: 27 - DNA, 20 bp, synthetic construct
  source 1..20: mol_type = other DNA
  modified_base 1..20: mod_base = OTHER; note = phosphorothioate internucleotide linkage
ctcaaccataacatgaccat

SEQ ID NO: 28 - DNA, 21 bp, synthetic construct
  source 1..21: mol_type = other DNA
  modified_base 1..21: mod_base = OTHER; note = phosphorothioate internucleotide linkage
ttaaagttcgtttagagaaca

SEQ ID NO: 29 - DNA, 20 bp, synthetic construct
  source 1..20: mol_type = other DNA
  modified_base 1..20: mod_base = OTHER; note = phosphorothioate internucleotide linkage
tactagtgcctgtgccgcac

SEQ ID NO: 30 - DNA, 20 bp, synthetic construct
  source 1..20: mol_type = other DNA
  modified_base 1..20: mod_base = OTHER; note = phosphorothioate internucleotide linkage
ggaaggtataaacctttaat

SEQ ID NO: 31 - DNA, 20 bp, synthetic construct
  source 1..20: mol_type = other DNA
  modified_base 1..20: mod_base = OTHER; note = phosphorothioate internucleotide linkage
ctgggaaggtataaaccttt

SEQ ID NO: 32 - DNA, 20 bp, synthetic construct
  source 1..20: mol_type = other DNA
  modified_base 1..20: mod_base = OTHER; note = phosphorothioate internucleotide linkage
caagagatcgaaagttggtt

SEQ ID NO: 33 - DNA, 20 bp, synthetic construct
  source 1..20: mol_type = other DNA
  modified_base 1..20: mod_base = OTHER; note = phosphorothioate internucleotide linkage
cagccacacagattttaaag

SEQ ID NO: 34 - DNA, 20 bp, synthetic construct
  source 1..20: mol_type = other DNA
  modified_base 1..20: mod_base = OTHER; note = phosphorothioate internucleotide linkage
ccactaagacaaacactaca

SEQ ID NO: 35 - DNA, 20 bp, synthetic construct
  source 1..20: mol_type = other DNA
  modified_base 1..20: mod_base = OTHER; note = phosphorothioate internucleotide linkage
cccacaatttagatgatgat

SEQ ID NO: 36 - DNA, 20 bp, synthetic construct
  source 1..20: mol_type = other DNA
  modified_base 1..20: mod_base = OTHER; note = phosphorothioate internucleotide linkage
caacggcagtacagacaaca

SEQ ID NO: 37 - DNA, 20 bp, synthetic construct
  source 1..20: mol_type = other DNA
  modified_base 1..20: mod_base = OTHER; note = phosphorothioate internucleotide linkage
cacaactacagccataacct

SEQ ID NO: 38 - DNA, 20 bp, synthetic construct
  source 1..20: mol_type = other DNA
  modified_base 1..20: mod_base = OTHER; note = phosphorothioate internucleotide linkage
taggttacctgtaaaacccc

SEQ ID NO: 39 - DNA, 20 bp, synthetic construct
  source 1..20: mol_type = other DNA
  modified_base 1..20: mod_base = OTHER; note = phosphorothioate internucleotide linkage
ctacatcaacaccatcaact

SEQ ID NO: 40 - DNA, 20 bp, synthetic construct
  source 1..20: mol_type = other DNA
  modified_base 1..20: mod_base = OTHER; note = phosphorothioate internucleotide linkage
gagacaaagtcattaagatc

SEQ ID NO: 41 - DNA, 20 bp, synthetic construct
  source 1..20: mol_type = other DNA
  modified_base 1..20: mod_base = OTHER; note = phosphorothioate internucleotide linkage
agaagagataaaatcatatc

SEQ ID NO: 42 - DNA, 20 bp, synthetic construct
  source 1..20: mol_type = other DNA
  modified_base 1..20: mod_base = OTHER; note = phosphorothioate internucleotide linkage
aagaagagtcacagtttgct

SEQ ID NO: 43 - DNA, 20 bp, synthetic construct
  source 1..20: mol_type = other DNA
  modified_base 1..20: mod_base = OTHER; note = phosphorothioate internucleotide linkage
cgtaaacggaaaagcgaaaa

SEQ ID NO: 44 - DNA, 19 bp, synthetic construct
  source 1..19: mol_type = other DNA
  modified_base 1..19: mod_base = OTHER; note = phosphorothioate internucleotide linkage
cattctcctaagaagctat

SEQ ID NO: 45 - DNA, 20 bp, synthetic construct
  source 1..20: mol_type = other DNA
  modified_base 1..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 46 - DNA, 20 bp, synthetic construct
  source 1..20: mol_type = other DNA
  modified_base 1..4: mod_base = OTHER; note = locked nucleic acid
  modified_base 17..20: mod_base = OTHER; note = locked nucleic acid
agtttcatcagccttcttct

SEQ ID NO: 47 - DNA, 20 bp, synthetic construct
  source 1..20: mol_type = other DNA
  modified_base 1..4: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 17..20: mod_base = OTHER; note = locked nucleic acid
agtttcatcagccttcttct

SEQ ID NO: 48 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = locked nucleic acid
  modified_base 17..20: mod_base = OTHER; note = locked nucleic acid
agtttcatcagccttcttct

SEQ ID NO: 49 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = locked nucleic acid
  modified_base 17..20: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 50 - DNA, 20 bp, synthetic construct
  source 1..20: mol_type = other DNA
  modified_base 1..4: mod_base = OTHER; note = locked nucleic acid
  modified_base 18..20: mod_base = OTHER; note = locked nucleic acid
agtttcatcagccttcttct

SEQ ID NO: 51 - DNA, 20 bp, synthetic construct
  source 1..20: mol_type = other DNA
  modified_base 1..4: mod_base = OTHER; note = locked nucleic acid
  modified_base 18..20: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 52 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = locked nucleic acid
  modified_base 18..20: mod_base = OTHER; note = locked nucleic acid
agtttcatcagccttcttct

SEQ ID NO: 53 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = locked nucleic acid
  modified_base 18..20: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 54 - DNA, 20 bp, synthetic construct
  source 1..20: mol_type = other DNA
  modified_base 1..3: mod_base = OTHER; note = locked nucleic acid
  modified_base 17..20: mod_base = OTHER; note = locked nucleic acid
agtttcatcagccttcttct

SEQ ID NO: 55 - DNA, 20 bp, synthetic construct
  source 1..20: mol_type = other DNA
  modified_base 1..3: mod_base = OTHER; note = locked nucleic acid
  modified_base 17..20: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 56 - DNA, 20 bp, synthetic construct
  source 1..20: mol_type = other DNA
  modified_base 1..20: mod_base = OTHER; note = phosphorothioate internucleotide linkage
  modified_base 1..3: mod_base = OTHER; note = locked nucleic acid
  modified_base 17..20: mod_base = OTHER; note = locked nucleic acid
agtttcatcagccttcttct

SEQ ID NO: 57 - DNA, 20 bp, synthetic construct
  source 1..20: mol_type = other DNA
  modified_base 1..20: mod_base = OTHER; note = phosphorothioate internucleotide linkage
  modified_base 1..3: mod_base = OTHER; note = locked nucleic acid
  modified_base 17..20: mod_base = OTHER; note = locked nucleic acid
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 58 - DNA, 20 bp, synthetic construct
  source 1..20: mol_type = other DNA
  modified_base 1..20: mod_base = OTHER; note = 2'-O-methoxyethyl
agtttcatcagccttcttct

SEQ ID NO: 59 - DNA, 20 bp, synthetic construct
  source 1..20: mol_type = other DNA
  modified_base 1..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 60 - DNA, 20 bp, synthetic construct
  source 1..20: mol_type = other DNA
  modified_base 1..20: mod_base = OTHER; note = 2'-O-methoxyethyl
  modified_base 1..20: mod_base = OTHER; note = phosphorothioate internucleotide linkage
agtttcatcagccttcttct

SEQ ID NO: 61 - DNA, 20 bp, synthetic construct
  source 1..20: mol_type = other DNA
  modified_base 1..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1..20: mod_base = OTHER; note = phosphorothioate internucleotide linkage
agtttcatcagccttcttct

SEQ ID NO: 62 - DNA, 20 bp, synthetic construct
  source 1..20: mol_type = other DNA
  modified_base 1..20: mod_base = OTHER; note = 2'-O-methoxyethyl
  modified_base 1..6: mod_base = OTHER; note = phosphorothioate internucleotide linkage
  modified_base 15..20: mod_base = OTHER; note = phosphorothioate internucleotide linkage
agtttcatcagccttcttct

SEQ ID NO: 63 - DNA, 20 bp, synthetic construct
  source 1..20: mol_type = other DNA
  modified_base 1..20: mod_base = OTHER; note = 2'-O-methoxyethyl
  modified_base 1..6: mod_base = OTHER; note = phosphorothioate internucleotide linkage
  modified_base 15..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 64 - DNA, 20 bp, synthetic construct
  source 1..20: mol_type = other DNA
  modified_base 1..20: mod_base = OTHER; note = 2'-O-methoxyethyl
  modified_base 1..6: mod_base = OTHER; note = phosphorothioate internucleotide linkage
  modified_base 16..20: mod_base = OTHER; note = phosphorothioate internucleotide linkage
agtttcatcagccttcttct

SEQ ID NO: 65 - DNA, 20 bp, synthetic construct
  source 1..20: mol_type = other DNA
  modified_base 1..20: mod_base = OTHER; note = 2'-O-methoxyethyl
  modified_base 1..6: mod_base = OTHER; note = phosphorothioate internucleotide linkage
  modified_base 16..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 66 - DNA, 20 bp, synthetic construct
  source 1..20: mol_type = other DNA
  modified_base 1..20: mod_base = OTHER; note = 2'-O-methoxyethyl
  modified_base 1..5: mod_base = OTHER; note = phosphorothioate internucleotide linkage
  modified_base 15..20: mod_base = OTHER; note = phosphorothioate internucleotide linkage
agtttcatcagccttcttct

SEQ ID NO: 67 - DNA, 20 bp, synthetic construct
  source 1..20: mol_type = other DNA
  modified_base 1..20: mod_base = OTHER; note = 2'-O-methoxyethyl
  modified_base 1..5: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 15..20: mod_base = OTHER; note = phosphorothioate internucleotide linkage
agtttcatcagccttcttct

SEQ ID NO: 68 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
agtttcatcagccttcttct

SEQ ID NO: 69 - DNA, 20 bp, synthetic construct
  source 1..20: mol_type = other DNA
  modified_base 1..5: mod_base = OTHER; note = 2'-O-methoxyethyl
  modified_base 16..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 70 - DNA, 20 bp, synthetic construct
  source 1..20: mol_type = other DNA
  modified_base 1..20: mod_base = OTHER; note = phosphorothioate internucleotide linkage
  modified_base 1..5: mod_base = OTHER; note = 2'-O-methoxyethyl
  modified_base 16..20: mod_base = OTHER; note = 2'-O-methoxyethyl
agtttcatcagccttcttct

SEQ ID NO: 71 - DNA, 20 bp, synthetic construct
  source 1..20: mol_type = other DNA
  modified_base 1..20: mod_base = OTHER; note = phosphorothioate internucleotide linkage
  modified_base 1..5: mod_base = OTHER; note = 2'-O-methoxyethyl
  modified_base 16..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 72 - DNA, 20 bp, synthetic construct
  source 1..20: mol_type = other DNA
  modified_base 1..5: mod_base = OTHER; note = 2'-O-methoxyethyl
  modified_base 17..20: mod_base = OTHER; note = 2'-O-methoxyethyl
agtttcatcagccttcttct

SEQ ID NO: 73 - DNA, 20 bp, synthetic construct
  source 1..20: mol_type = other DNA
  modified_base 1..5: mod_base = OTHER; note = 2'-O-methoxyethyl
  modified_base 17..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 74 - DNA, 20 bp, synthetic construct
  source 1..20: mol_type = other DNA
  modified_base 1..20: mod_base = OTHER; note = phosphorothioate internucleotide linkage
  modified_base 1..5: mod_base = OTHER; note = 2'-O-methoxyethyl
  modified_base 17..20: mod_base = OTHER; note = 2'-O-methoxyethyl
agtttcatcagccttcttct

SEQ ID NO: 75 - DNA, 20 bp, synthetic construct
  source 1..20: mol_type = other DNA
  modified_base 1..20: mod_base = OTHER; note = phosphorothioate internucleotide linkage
  modified_base 1..5: mod_base = OTHER; note = 2'-O-methoxyethyl
  modified_base 17..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 76 - DNA, 20 bp, synthetic construct
  source 1..20: mol_type = other DNA
  modified_base 1..4: mod_base = OTHER; note = 2'-O-methoxyethyl
  modified_base 16..20: mod_base = OTHER; note = 2'-O-methoxyethyl
agtttcatcagccttcttct

SEQ ID NO: 77 - DNA, 20 bp, synthetic construct
  source 1..20: mol_type = other DNA
  modified_base 1..4: mod_base = OTHER; note = 2'-O-methoxyethyl
  modified_base 16..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 78 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = 2'-O-methoxyethyl
  modified_base 16..20: mod_base = OTHER; note = 2'-O-methoxyethyl
agtttcatcagccttcttct

SEQ ID NO: 79 - DNA, 20 bp, synthetic construct
  source 1..20: mol_type = other DNA
  modified_base 1..20: mod_base = OTHER; note = phosphorothioate internucleotide linkage
  modified_base 1..4: mod_base = OTHER; note = 2'-O-methoxyethyl
  modified_base 16..20: mod_base = OTHER; note = 2'-O-methoxyethyl
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 80 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 81 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..5: mod_base = OTHER; note = 2'-O-methyl modification
  modified_base 7..8: mod_base = OTHER; note = 2'-O-methyl modification
  modified_base 6: mod_base = cm
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 82 - DNA, 20 bp, synthetic construct
  source 1..20: mol_type = other DNA
  modified_base 1..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 83 - DNA, 20 bp, synthetic construct
  source 1..20: mol_type = other DNA
  modified_base 1..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 84 - DNA, 20 bp, synthetic construct
  source 1..20: mol_type = other DNA
  modified_base 1..6: mod_base = OTHER; note = phosphorothioate internucleotide linkage
  modified_base 15..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 85 - DNA, 20 bp, synthetic construct
  source 1..20: mol_type = other DNA
  modified_base 1..6: mod_base = OTHER; note = phosphorothioate internucleotide linkage
  modified_base 15..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 86 - DNA, 20 bp, synthetic construct
  source 1..20: mol_type = other DNA
  modified_base 1..6: mod_base = OTHER; note = phosphorothioate internucleotide linkage
  modified_base 16..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 87 - DNA, 20 bp, synthetic construct
  source 1..20: mol_type = other DNA
  modified_base 1..6: mod_base = OTHER; note = phosphorothioate internucleotide linkage
  modified_base 16..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 88 - DNA, 20 bp, synthetic construct
  source 1..20: mol_type = other DNA
  modified_base 1..5: mod_base = OTHER; note = phosphorothioate internucleotide linkage
  modified_base 15..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 9: mod_base = cm
  modified_base 10: mod_base = OTHER; note = 2'-O-methyl modification
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 89 - DNA, 20 bp, synthetic construct
  source 1..20: mol_type = other DNA
  modified_base 1..5: mod_base = OTHER; note = phosphorothioate internucleotide linkage
  modified_base 15..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 6: mod_base = cm
  modified_base 7..8: mod_base = OTHER; note = 2'-O-methyl modification
  modified_base 10: mod_base = OTHER; note = 2'-O-methyl modification
  modified_base 9: mod_base = cm
  modified_base 11: mod_base = gm
  modified_base 12..13: mod_base = cm
  modified_base 14..15: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 90 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..5: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 91 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..5: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
  modified_base 6: mod_base = m5c
  modified_base 9: mod_base = m5c
  modified_base 12..13: mod_base = m5c
  modified_base 16: mod_base = m5c
  modified_base 19: mod_base = m5c
agtttcatcagccttcttct

SEQ ID NO: 92 - DNA, 20 bp, synthetic construct
  source 1..20: mol_type = other DNA
  modified_base 1..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 93 - DNA, 20 bp, synthetic construct
  source 1..20: mol_type = other DNA
  modified_base 1..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 94 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..5: mod_base = OTHER; note = 2'-O-methyl modification
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 95 - DNA, 20 bp, synthetic construct
  source 1..20: mol_type = other DNA
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 96 - DNA, 20 bp, synthetic construct
  source 1..20: mol_type = other DNA
  modified_base 1..20: mod_base = OTHER; note = phosphorothioate internucleotide linkage
  modified_base 1: mod_base = OTHER; note = 2'-O-methyl modification
  modified_base 2: mod_base = gm
  modified_base 3..5: mod_base = OTHER; note = 2'-O-methyl modification
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 97 - DNA, 20 bp, synthetic construct
  source 1..20: mol_type = other DNA
  modified_base 1..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..5: mod_base = OTHER; note = 2'-O-methyl modification
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 98 - DNA, 20 bp, synthetic construct
  source 1..20: mol_type = other DNA
  modified_base 1: mod_base = OTHER; note = 2'-O-methyl modification
  modified_base 2: mod_base = gm
  modified_base 3..4: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 99 - DNA, 20 bp, synthetic construct
  source 1..20: mol_type = other DNA
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..4: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 100 - DNA, 20 bp, synthetic construct
  source 1..20: mol_type = other DNA
  modified_base 1..20: mod_base = OTHER; note = phosphorothioate internucleotide linkage
  modified_base 1: mod_base = OTHER; note = 2'-O-methyl modification
  modified_base 2: mod_base = gm
  modified_base 3..4: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 101 - DNA, 20 bp, synthetic construct
  source 1..20: mol_type = other DNA
  modified_base 1..20: mod_base = OTHER; note = phosphorothioate internucleotide linkage
  modified_base 6: mod_base = m5c
  modified_base 9: mod_base = m5c
  modified_base 12..13: mod_base = m5c
  modified_base 16: mod_base = m5c
  modified_base 19: mod_base = m5c
  modified_base 1: mod_base = OTHER; note = 2'-O-methyl modification
  modified_base 2: mod_base = gm
  modified_base 3..4: mod_base = OTHER; note = 2'-O-methyl modification
  modified_base 16: mod_base = cm
  modified_base 17..18: mod_base = OTHER; note = 2'-O-methyl modification
  modified_base 19: mod_base = cm
  modified_base 20: mod_base = OTHER; note = 2'-O-methyl modification
agtttcatcagccttcttct

SEQ ID NO: 102 - DNA, 20 bp, synthetic construct
  source 1..20: mol_type = other DNA
  modified_base 1..20: mod_base = OTHER; note = phosphorodiamidate morpholino oligomer
agtttcatcagccttcttct

SEQ ID NO: 103 - DNA, 20 bp, synthetic construct
  source 1..20: mol_type = other DNA
  modified_base 1..20: mod_base = OTHER; note = peptide nucleic acid
agtttcatcagccttcttct

SEQ ID NO: 104 - DNA, 3242 bp, synthetic construct
  source 1..3242: mol_type = other DNA
taatacgactcactatagattaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggtcaccgacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatggg

SEQ ID NO: 105 - DNA, 20 bp, synthetic construct
  source 1..20: mol_type = other DNA
  modified_base 1..12: mod_base = OTHER; note = phosphorothioate internucleotide linkage
  modified_base 13..20: mod_base = OTHER; note = phosphorothioate internucleotide linkage
  modified_base 1..2: mod_base = OTHER; note = 2'-O-methyl modification
  modified_base 6..7: mod_base = OTHER; note = 2'-O-methyl modification
  modified_base 9..13: mod_base = OTHER; note = 2'-O-methyl modification
  modified_base 16: mod_base = OTHER; note = 2'-O-methyl modification
  modified_base 19: mod_base = OTHER; note = 2'-O-methyl modification
agtttcatcagccttcttct

SEQ ID NO: 106 - RNA, 22 nt, synthetic construct
  source 1..22: mol_type = other RNA
  modified_base 20..22: mod_base = OTHER; note = thymine
gaagaaggctgatgaaactttt

SEQ ID NO: 107 - RNA, 21 nt, synthetic construct
  source 1..21: mol_type = other RNA
  modified_base 20..21: mod_base = OTHER; note = thymine
agtttcatcagccttcttctt